(19) United States
(12) Patent Application Publication
(10) Pub. No.: US 2021/0074401 A1
(43) Pub. Date: Mar. 11, 2021

(54) METHODS AND SYSTEMS TO PROVIDE DRUG PRICING INFORMATION ACCORDING TO CUSTOMER PROFILE

(71) Applicant: GoodRx, Inc., Santa Monica, CA (US)

(72) Inventors: Trevor Zachary Bezdek, Los Angeles, CA (US); Douglas Joseph Hirsch, Los Angeles, CA (US); Andrew David Slutsky, Los Angeles, CA (US); Gracye Yardwin Cheng, Santa Monica, CA (US); Justin Charles Fengler, Santa Monica, CA (US)

(21) Appl. No.: 16/995,105

(22) Filed: Aug. 17, 2020

Related U.S. Application Data
(63) Continuation of application No. 15/943,921, filed on Apr. 3, 2018, now abandoned.

(51) Int. Cl. G16H 20/10; G06Q 40/08; G16H 10/60

(52) U.S. Cl. CPC G16H 20/10; H04W 4/12; G16H 10/60; G06Q 40/08

(57) ABSTRACT

A drug pricing system receives a request for drug pricing from a pharmacy comprised in part of a unique set of identifiers that routes the drug pricing request to the drug pricing system, obtains drug prices from a user's insurance company based on the user's insurance information, obtains drug prices from multiple pharmacy benefit managers, and determines a lowest drug price. The drug pricing system sends a digital alert to a user interface when the user prefers to use the insurance price and the insurance price is more than the lower PBM price. In response to the digital alert, the drug pricing system receives a response from the user interface that includes an updated user preference, which the drug pricing system stores in a user profile. The drug pricing system returns to the pharmacy the drug price according to the updated user preference.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

This invention relates to managing medical benefits and, more particularly, to managing pharmacy benefits to reduce costs.

Health care costs in the United States have risen dramatically over the past several decades. Pharmacy Benefit Managers (PBMs) contract with pharmacies to provide prescriptions at negotiated prices to patients who have access to the PBMs' rates. When patients purchase prescriptions using these negotiated prices, the applicable PBM then processes these prescription claims. PBMs' manage the negotiated rates at a pharmacy across different sets of rates that the PBM provides to different clients—in most cases, benefit providers. PBMs are typically entities that are independent of the benefit provider, e.g. an insurance company, and contract with the benefit provider such that the benefit provider can access a set of rates provided by the PBM and to process claims under the applicable set of rates. In addition to providing prices for benefit providers and processing insurance claims, PBMs also provide prices to and process claims for “unfunded” discount programs, which provide patients access to a set of PBM's negotiated prices at pharmacies outside of an insurance benefit.

The distribution channels for prescription drugs are, in many cases, separated from the payment channels. For example, a patient may be diagnosed by a physician as having a condition that requires medication. The physician then decides on a drug appropriate for treatment of the diagnosed condition and prepares a prescription for an appropriate drug. The patient then takes the prescription to a pharmacy for dispensing of the prescription drugs. If the patient has a prescription drug benefit, e.g., through health insurance coverage, the pharmacist will utilize their computer system to access the PBMs computer system to apply the negotiated charge. Consequently, the patient or prescriber may not be aware of the differing costs for the drug by different PBMs and/or at different pharmacies, or that the patient may also utilize a discount program.

Furthermore, different PBMs operate under different agreements with pharmacies. For example, the price for a drug associated with one PBM can often differ significantly with respect to a second PBM. Accordingly, one PBM may provide a lower cost on one drug than other PBMs, but have a much higher cost for other drugs. In addition, the price for a drug at one pharmacy using a given PBM will differ from the price provided by that same PBM at another pharmacy. In addition, PBMs offer different sets of rates, such that, even with the same PBM, the prices provided to different insurance plans will vary between plans, and will be different than the rates provided via an unfunded discount program. Therefore, across PBMs, prices provided via an insurance plan may actually be higher than prices provided by a discount program.

Many Americans assume that the way to manage prescription costs is simply to obtain pay for health insurance or Medicare (thereby utilizing a particular PBM network provided to an insurance plan), and then show up at any pharmacy counter. While this may have been true years ago, it is no longer reality. Insurance companies are increasing prescription drug deductibles and patient co-pays while reducing the numbers and quantities of the drugs that they will pay for. Even patients with insurance may find that their specific prescriptions are not covered. Meanwhile, hundreds of medicines can be purchased using an unfunded discount program for less than an insurance co-pay.

### SUMMARY

In order to address these and other challenges, a system according to certain aspects of the disclosure provides drug pricing information from multiple PBMs to users. For example, the system may obtain, calculate, and/or estimate drug prices that are available under contracts or agreements between PBMs and various pharmacies. These prices may be prices of drugs for purchase at the various pharmacies. The system can process the drug pricing information such that it can be readily provided to users in response to requests for prices of particular drugs. In response to such requests, the system can display relevant prices. For example, the system displays a price for each pharmacy chain and/or displays prices for a particular geographical area. The users can compare the prices for a particular drug and determine which pharmacy they would like to purchase the drug from. The system can provide a discount coupon that allows the users to purchase the drug at the prices listed by the system at any of the listed pharmacies. As part of the provided discount coupon, the system provides the user with a unique set of identifiers, including a BIN, which refers to a Bank Identification Number, specific to a system. The BIN allows a pharmacy's computer system to route the electronic claim to the system, which selects the appropriate PBM and PBM fee schedule to process the electronic claim and then routes the electronic claim to a specific PBM and PBM fee schedule to be processed at the selected price.

The system can aggregate drug pricing information from multiple PBMs and present the data to the users in a simple and easy-to-digest manner. As such, the system can provide users with convenience and valuable information that can translate into savings in time and costs. The system may provide a portion of the aggregated drug pricing information and a unique set of identifiers, including the BIN associated with the system, to the user.

In one embodiment of the inventive system, the system identifies the lower-cost pharmacy for a particular drug. A user enters information about a prescription such as the name of the drug (generic or brand-name), the form and the dosage. The user also provides or the system detects a location (city, state or ZIP), and the system identifies the prices the user can obtain at local and mail order pharmacies for a variety of dosages and quantities for that prescription.

The system searches the fee schedules associated with multiple PBMs to identify the drug pricing information. In one embodiment, the system contracts with multiple PBMs such that the system can pass the PBM savings onto the users. The users do not need to contract directly with the PBMs. Rather, the system is associated with multiple PBMs, displays prices for drugs from a plurality of PBMs and provides the appropriate PBM discount for a particular pharmacy. The PBM prices accessed by the system may be unfunded rates that can be utilized by any users and do not require participation in an insurance plan.

In an embodiment, the system provides the user with a portion of the identified drug prices and a unique set of identifiers, including a BIN specific to the system. In this instance, users do not have to register to obtain the unique set of identifiers. In an embodiment, the user can provide the unique set of identifiers to a pharmacist and the pharmacist can enter the unique set of identifiers into the pharmacy's computer system. The user's electronic claim is then routed to the system, which selects the appropriate PBM and PBM fee schedule to process the electronic claim and then routes the electronic claim to a specific PBM and PBM fee schedule to be processed at the selected price.

In one embodiment, the system works with the multiple PBMs and a insurance pricing provider, which may be a PBM, a health insurance company or other third party provider of insurance pricing information. In this instance, users may register for a user profile to store their insurance information. In addition to entering information about the prescription and a location, the user enters health insurance information, such as, but not limited to the provider name, group, issuer number, member ID. The user may enter the health insurance information by typing the information in or providing a photo of their insurance card. The system creates a user profile and stores the user identification information, user insurance information and user prescriptions in the user profile. The system identifies whether a prescription is covered by the prescription benefit plan and the prices the user can obtain at local and mail order pharmacies with the prescription benefit plan associated with the user's health insurance for a variety of dosages and quantities for that prescription. The system identifies the unfunded rates the user can obtain without insurance at local and mail order pharmacies using the system's sets of prices at multiple PBMs for a variety of dosages and quantities for that prescription. The system can aggregate unfunded drug pricing information from multiple PBMs and insurance prices from the insurance pricing provider associated with the user's health insurance and present the data to the user.

The system may provide a portion of the insurance drug pricing information, PBM rates and unique set of identifiers, including the BIN associated with the system to the user. In an embodiment, the user can provide the unique set of identifiers to a pharmacist and the pharmacist can enter the unique set of identifiers into the pharmacy's computer system. The user's electronic claim is then routed to the system, which may route the electronic claim to the insurance pricing provider to be processed at the insurance price for that pharmacy, or select the appropriate PBM and PBM fee schedule to process the electronic claim at an unfunded rate and then routes the electronic claim to a specific PBM and PBM fee schedule to be processed at the selected price at that pharmacy. In some embodiments, this may result in a reversal by the system to the PBM.

In one embodiment, the user registers with the system. The user provides identification information. In another The user may also provide health insurance information and an indication as to whether the user prefers to fill prescriptions using the prescription benefit plan associated with the health insurance or prefers to fill prescriptions at a lower PBM rate provided by the system, when available. The system creates a user profile and stores the user identification information, user insurance information and preferences in the user profile. The system may show to the user a discount card with a set of unique identifiers that can be presented at a pharmacy. The pharmacy enters the information from the discount card into the pharmacy computer system. The BIN provided on the discount card routes the message from the pharmacy to the system. The system retrieves and aggregates drug pricing information from multiple PBMs and from the insurance pricing provider.

In an embodiment, if no insurance information is provided, the system returns a PBM rate from to the pharmacy. If insurance information is provided, the system compares the insurance price with the lower PBM rate from the aggregated PBM drug pricing information and returns a rate to the pharmacy based on the user preference specified in the user profile. In an embodiment, the lower PBM rate is the overall lowest rate available from a PBM. In another embodiment, the lower PBM rate is a lower PBM rate. In another embodiment, other factors, such as, but not limited to accuracy, may influence what price is returned as the lower PBM rate. The lower PBM rate returned may be a drug price that is more than the absolute lowest available PBM rate.

If the user profile indicates that the user prefers the lower-cost drug price, the system returns to the pharmacy the lower of the insurance price or the lower PBM rate from the aggregated PBM drug pricing information. If the user profile indicates that the user prefers to use insurance, the system returns the health insurance rate to the pharmacy. If the user has not selected a preference, the system may default to either the health insurance rate or the lower PBM rate.

In an embodiment, if the user profile indicates that the user prefers to fill prescriptions using the prescription benefit plan of the insurance company and if the system determines that the aggregated PBM drug pricing information has a price that is lower than the insurance price, the system sends a digital alert to the user. The digital alert, such as a text message, SMS message, email, or the like, informs the user that the system has found a lower price for the drug without using the insurance and asks if the user wants to utilize the lower price without using insurance for the current fill of the prescription and/or future fills of the prescription, or if the user wants to continue filling the prescription using the insurance. The user selects a preference via a user interface and the system saves the user preference and updates the user profile. If the user profile is updated by the system within a specified time period (approximating a time period where the user is likely still at the pharmacy), the system will return the unfunded rate without insurance to the pharmacy for the current fill. If the user profile is not updated within a specified time period, the insurance price for the prescription is returned to the pharmacy, and the user profile is updated such that, for future fills of the prescription, the system will return the unfunded rate without insurance to the pharmacy.

According to one aspect, the present disclosure relates to a method to provide drug pricing to a pharmacy system for a drug requested by the user in a drug pricing environment. The drug pricing environment can have a drug pricing system, one or more PBMs, an insurance pricing system, and a pharmacy system. The drug pricing system comprises one or more hardware processors and memory including drug pricing information and user profiles of users associated with the drug pricing system. The method is implemented by the one or more hardware processors configured to execute specific instructions stored in the memory and comprises receiving from the pharmacy system a transmission comprising at least a routing identifier identifying the drug pricing system as a recipient of the transmission and a request for the drug pricing of the drug. The transmission may also include a user identifier. In some embodiments, this may result in a reversal by the system to the PBM.

The method further comprises obtaining a first set of PBM prices, which may be unfunded prices, for the drug from a first PBM, obtaining a second set of PBM prices, which may be unfunded prices, for the drug from a second PBM, determining the lower PBM rate based at least in part on the first and second sets of PBM prices for the drug, and retrieving, if available, the user profile of the user using the user identifier. The user profile may comprise of insurance information of the user and a user pricing preference indicating whether the user prefers to use an insurance price or a lower PBM rate for the drug, if available.

The method further comprises obtaining the insurance price from the insurance pricing system based on the insurance information of the user, determining the lower drug price for the drug based at least in part on the insurance price and the lower PBM rate, and sending a digital alert comprising at least the lower drug price to a user interface to notify the user when the user pricing preference indicates that the user prefers to use the insurance price and the insurance price is greater than the lower PBM rate. The method further comprises receiving from the user interface an updated user preference, updating the user profile with the updated user preference, and returning to the pharmacy system the drug pricing according to the updated user preference. The user preference may be used for the current fill of the prescription and/or for subsequent fills of the prescription.

In some embodiments, the method further comprises storing one or more of the first set of PBM prices, one or more of the second set of PBM prices, the lower PBM rate at each pharmacy, the insurance price at each pharmacy (insurance prices can vary by pharmacy) and the lower of the lower PBM rate and the insurance price at each pharmacy.

In certain embodiments, the method further comprises reformatting the lower PBM rate and/or the insurance price that is stored in the memory to a format usable by the digital alert.

In various embodiments, the method further comprises reformatting the updated user preference received from the user interface to a format usable by the memory for storage in the user profile.

In an embodiment, the digital alert is a text message or an email and the user interface is a mobile device.

In another embodiment, the user profile further comprises a user contact preference indicating how the user prefers to receive the digital alert.

In some embodiments, the method further comprises sending the digital alert in accordance with the user contact preference.

In a further embodiment, the routing identifier is a National Council for Prescription Drug Programs (NCPDP) Processor ID Number. In certain embodiments, the routing identifier includes a Group Number and a Member ID.

In an embodiment, the routing identifier is one of a Bank Identification Number (BIN) and an Issuer Identification Number (IIN).

In certain embodiments, the method further comprises transmitting the drug pricing to the pharmacy system in accordance with a National Council for Prescription Drug Programs (NCPDP) standards for electronic drug claims.

In certain embodiments, the present disclosure relates to a drug pricing system for use in a drug pricing environment that has one or more PBMs, an insurance pricing system, and a pharmacy system. The drug pricing system comprises one or more hardware processors and memory including drug pricing information and user profiles of users associated with the drug pricing system. The one or more hardware processors are configured to receive from the pharmacy system a transmission comprising at least a routing identifier identifying the drug pricing system as a recipient of the transmission, a request for the drug pricing of the drug. The transmission may also include a user identifier. If the user has created a user profile in the system, the user identifier will identify the user in the drug pricing system.

The one or more hardware processors of the drug pricing system are further configured to obtain a first set of PBM prices, which may be unfunded prices, for the drug from a first PBM, obtain a second set of PBM prices, which may be unfunded prices, for the drug from a second PBM, and determine the lower PBM rate based at least in part on the first and second sets of PBM prices for the drug. The one or more hardware processors of the drug pricing system are further configured to retrieve, based on the user identifier, the user profile of the user when available. The user profile may comprise of insurance information of the user and a user pricing preference indicating whether the user prefers to use an insurance price or a lower PBM price for the drug.

The one or more hardware processors of the drug pricing system are further configured to obtain the insurance price from the insurance pricing system based on the insurance information of the user, determine if there is a lower PBM rate, and, if so, send a digital alert comprising at least the lower drug price to a user interface to notify the user when the user pricing preference indicates that the user prefers to use the insurance price and the insurance price is greater than the lower PBM rate, receive from the user interface an updated user preference, update the user profile with the updated user preference, and return to the pharmacy system the drug pricing according to the updated user preference.

In some embodiments, the one or more hardware processor are further configured to store the first set of PBM prices, one or more of the second set of PBM prices, the lower PBM rate at each pharmacy, the insurance price at each pharmacy (insurance prices can vary by pharmacy) and the lower of the lower PBM rate and the insurance price at each pharmacy.

In certain embodiments, the one or more hardware processors are further configured to reformat the lower PBM rate and/or the insurance price that is stored in the memory to a format usable by the digital alert.

In some embodiments, the one or more hardware processors are further configured to reformat the updated user preference received from the user interface to a format usable by the memory for storage in the user profile.

In certain embodiments, the digital alert is a text message or push notification and the user interface is a mobile device.

In some embodiments, the user profile further comprises a user contact preference indicating how the user prefers to receive the digital alert.

In some embodiments, the one or more hardware processors are further configured to send the digital alert in accordance with the user contact preference.

In certain embodiments, the routing identifier is a National Council for Prescription Drug Programs (NCPDP) Processor ID Number. In certain embodiments, the routing identifier includes a Group Number and a Member ID.

In some embodiments, the routing identifier is one of a Bank Identification Number (BIN) and an Issuer Identification Number (IIN).

In another embodiment, the one or more hardware processor are further configured to transmit the drug pricing to the pharmacy system in accordance with a National Council for Prescription Drug Programs (NCPDP) standards for electronic drug claims.

According to one aspect, the present disclosure relates to a method to provide insured users drug pricing information from multiple drug pricing systems. The method is implemented by one or more hardware processors configured to execute specific instructions stored in memory and comprises the user creating a user profile, receiving from a user interface at least a request for drug pricing for a drug from a user and insurance information from or associated with a user, correlating a unique identifier assigned to the user and the insurance information in a user profile that is stored in the memory, obtaining through an interface, such as a first application programming interface (API), a first set of PBM prices for the drug from a first PBM, obtaining through an interface, such as a second API, a second set of PBM prices for the drug from a second PBM.

The method further comprises obtaining through an interface such as a third API an insurance price from an insurance pricing system based on the insurance information of the user, presenting in the user interface a unique set of identifiers (which may include a BIN associated specifically with the system, as well as a processor control number (PCN), group number and member ID), a portion of the first set of PBM prices, a portion of the second set of PBM prices, and a set of insurance prices, and receiving from the user interface the unique set of identifiers and an indication that the user elects to use insurance prices or a lower PBM rate, when available. The method further comprises correlating the indication with the user profile when the indication is received.

In some embodiments, each price in the first set of PBM prices is determined by an agreement between the first PBM and a pharmacy system. In some embodiments, each price in the second set of PBM prices determined by an agreement between the second PBM and a pharmacy system.

In various embodiments, the method further comprises determining that the drug is covered by the insurance pricing system in accordance with the user insurance information. In various embodiments, the method further comprises determining whether additional approvals and/or steps are necessary to obtain the drug and/or drug pricing, such as in the case of a drug that needs a prior authorization.

In certain embodiments, the unique set of identifiers comprises a first part identifying the drug pricing system and a second part identifying the user.

In an embodiment, the first and second sets of PBM prices comprise unfunded drug prices.

In some embodiments, the request for drug pricing includes one or more of a drug name, a drug form, a dosage, and a quantity.

In certain embodiments, obtaining the first set of PBM prices for the drug from the first PBM comprises performing a mock adjudication. In other embodiments, obtaining the first set of PBM prices for the drug from the first PBM comprises performing an actual adjudication.

In various embodiments, obtaining an insurance price from an insurance pricing system comprises performing a mock adjudication. In other embodiments, obtaining the insurance price for the drug from the insurance pricing system comprises performing an actual adjudication.

In an embodiment, the insurance information includes one or more of a user name, a name of the insurance pricing system, a group number, and a member ID. In some embodiments, the drug pricing system returns a message to the pharmacy.

In certain aspects, the present disclosure relates to a drug pricing system to provide insured users drug pricing information from multiple drug pricing sources. The drug pricing system comprises one or more hardware processors configured to execute specific instructions stored in memory. The one or more hardware processors are configured to receive from a user interface at least a request for drug pricing for a drug and insurance information from or associated with a user, correlate a unique set of identifiers assigned to the user and the insurance information in a user profile that is stored in the memory, obtain through an interface such as an API a first set of PBM prices for the drug from a first PBM, obtain through an interface such as an API a second set of PBM prices for the drug from a second PBM.

The one or more hardware processors of the drug pricing system are further configured to obtain through an interface such as an API an insurance price from an insurance pricing system based on the insurance information of the user, present in the user interface the unique set of identifiers, a portion of the first set of PBM prices, a portion of the second set of PBM prices, and a set of insurance prices, receive from the user interface one of the unique set of identifiers that are presented to the user and an indication that the user elects to use insurance, and correlate the indication with the user profile when the indication is received.

In some embodiments, the one or more hardware processors are further configured to determining that the drug is covered by the insurance pricing system in accordance with the user insurance information. In various embodiments, the method further comprises determining whether additional approvals and/or steps are necessary to obtain the drug and/or drug pricing, such as in the case of a drug that needs a prior authorization.

According to one aspect, the present disclosure relates to a method to provide drug pricing to the pharmacy system for a drug requested by the user in a drug pricing environment having a drug pricing system, one or more PBMs, an insurance pricing system, and a pharmacy system, the drug pricing system comprising one or more hardware processors and memory including drug pricing information and user profiles of users associated with the drug pricing system. The method comprises, as implemented by the one or more hardware processors configured to execute specific instructions stored in the memory, receiving from the pharmacy system a transmission comprising at least a routing identifier identifying the drug pricing system as a recipient of the transmission, a request for drug pricing of the drug, and a user identifier identifying the user in the drug pricing system; obtaining a first set of PBM prices for the drug from a first PBM; obtaining a second set of PBM prices for the drug from a second PBM; determining a lower PBM rate based at least in part on the first and second sets of PBM prices for the drug; retrieving, based on the user identifier, the user profile of the user, the user profile comprising at least insurance information of the user and a user pricing preference indicating whether the user prefers to use an insurance price or the lower PBM rate for the drug; obtaining the insurance price from the insurance pricing system based on the insurance information of the user; determining the lowest drug price for the drug based at least in part on the insurance price and the lower PBM rate; sending a digital alert comprising at least the lowest drug price to a user interface to notify the user when the user pricing preference indicates that the user prefers to use the insurance price and the insurance price is greater than the lower PBM rate; receiving from the user interface an updated user preference; updating the user profile with the updated user preference; and returning to the pharmacy system the drug pricing according to the updated user preference.

According to one aspect, the present disclosure relates to drug pricing system for use in a drug pricing environment having one or more PBMs, an insurance pricing system, and a pharmacy system. The drug pricing system comprises one or more hardware processors and memory including drug pricing information and user profiles of users associated with the drug pricing system, the one or more hardware processors configured to receive from the pharmacy system a transmission comprising at least a routing identifier identifying the drug pricing system as a recipient of the transmission, a request for drug pricing of the drug, and a user identifier identifying the user in the drug pricing system; obtain a first set of PBM prices for the drug from a first PBM; obtain a second set of PBM prices for the drug from a second PBM; determine a lower PBM rate based at least in part on the first and second sets of PBM prices for the drug; retrieve, based on the user identifier, the user profile of the user, the user profile comprising at least insurance information of the user and a user pricing preference indicating whether the user prefers to use an insurance price or a lower PBM rate for the drug; obtain the insurance price from the insurance pricing system based on the insurance information of the user; determine which of the insurance price and the lower PBM rate is the lowest drug price for the drug; send a digital alert comprising at least the lower PBM rate to a user interface to notify the user when the user pricing preference indicates that the user prefers to use the insurance price and the insurance price is greater than the lower PBM rate; receive from the user interface an updated user preference; update the user profile with the updated user preference; and return to the pharmacy system the drug pricing according to the updated user preference.

According to one aspect, the present disclosure relates to method to provide users drug pricing information from multiple drug pricing systems. The method comprises, as implemented by one or more hardware processors configured to execute specific instructions stored in memory, receiving from a user interface at least a request for drug pricing for a drug from or associated with a user and insurance information from or associated with a user; correlating a set of unique identifiers assigned to the user and the insurance information in a user profile that is stored in the memory; obtaining through a first API a first set of PBM prices for the drug from a first PBM; obtaining through a second API a second set of PBM prices for the drug from a second PBM; obtaining through a third API an insurance price from an insurance pricing system based on the insurance information of the user; presenting in the user interface the set of unique identifiers, a portion of the first set of PBM prices, a portion of the second set of PBM prices, and the insurance price; receiving from the user interface an indication of a user pricing preference; and correlating the indication with the user profile when the indication is received.

According to one aspect, the present disclosure relates to drug pricing system to provide users drug pricing information from multiple drug pricing sources. The drug pricing system comprises one or more hardware processors configured to execute specific instructions stored in memory, the one or more hardware processors configured to receive from a user interface at least a request for drug pricing for a drug and insurance information from or associated with a user; correlate a set of unique identifiers assigned to the user and the insurance information in a user profile that is stored in the memory; obtain through a first API a first set of PBM prices for the drug from a first PBM; obtain through a second API a second set of PBM prices for the drug from a second PBM; obtain through a third API an insurance price from an insurance pricing system based on the insurance information of the user; present in the user interface the set of unique identifiers, a portion of the first set of PBM prices, a portion of the second set of PBM prices, and the insurance price; receive from the user interface an indication of a user pricing preference; and correlate the indication with the user profile when the indication is received.

According to one aspect, the present disclosure relates to method to provide users drug pricing information from multiple drug pricing systems. The method comprises as implemented by one or more hardware processors configured to execute specific instructions stored in memory, receiving from a user interface at least a request for drug pricing for a drug from or associated with a user; obtaining through a first API a first set of PBM prices for the drug from a first PBM; obtaining through a second API a second set of PBM prices for the drug from a second PBM; and presenting in the user interface, a portion of the first set of PBM prices and a portion of the second set of PBM prices and a set of unique identifiers that will allow the user to receive similar pricing as the first and second sets of PBM prices for the drug when the set of unique identifiers are presented at a pharmacy system.

In an embodiment, the method further comprises obtaining prices for the drug from other than the first and second PBMs. In another embodiment, the method further comprises generating drug pricing requests periodically based on the user profile and presenting in the user interface at least one of a price for the drug and a message. In a further embodiment, the method further comprises determining benefit information associated with insurance benefits of the user in accordance with the insurance information of the user.

For purposes of summarizing the disclosure, certain aspects, advantages and novel features of the inventions have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

FIG. 1 illustrates an overview of an example system to provide drug prices, according aspects of the disclosure.

FIG. 2 illustrates an example data flow diagram to provide drug prices from multiple PBMs, according to aspects of the disclosure.

FIG. 3 is a flow chart illustrating an example process to display drug prices from multiple for PBMs, according to aspects of the disclosure.

FIG. 4 illustrates an example data flow diagram to provide drug prices from multiple PBMs and/or an insurance price provider, according to aspects of the disclosure.

FIG. 5 is a flow chart illustrating an example process to display drug prices from multiple PBMs and/or an insurance price provider, according to aspects of the disclosure.

FIG. 6 illustrates an example data flow diagram to provide drug prices in response to a user profile, according to aspects of the disclosure.

FIG. 7 is a flow chart illustrating an example process to generate a user profile, according to aspects of the disclosure.

FIGS. 8A and 8B are a flow chart illustrating an example process to provide drug prices in response to a user profile, according to aspects of the disclosure.

FIG. 9 illustrates an example discount card with the unique set of identifiers, according to aspects of the disclosure.

FIG. 10 illustrates an example of a user interface to provide drug prices from various PBMs and/or an insurance price provider, according to aspects of the disclosure.

FIG. 11 illustrates an example of a user interface to provide drug prices from various PBMs and/or an insurance price provider, according to aspects of the disclosure.

FIG. 12A illustrates an example of a digital alert provided to the user interface, according to aspects of the disclosure.

FIGS. 12B and 12C illustrate examples of user responses sent from the user interface, according to aspects of the disclosure.

### DETAILED DESCRIPTION

The disclosure provided in the following pages describes examples of some embodiments of the invention. The designs, figures, and description are non-limiting examples of some embodiments of the invention. Other embodiments of the system may or may not include the features disclosed herein. Moreover, disclosed advantages and benefits may apply to only some embodiments of the invention, and should not be used to limit the scope of the invention.

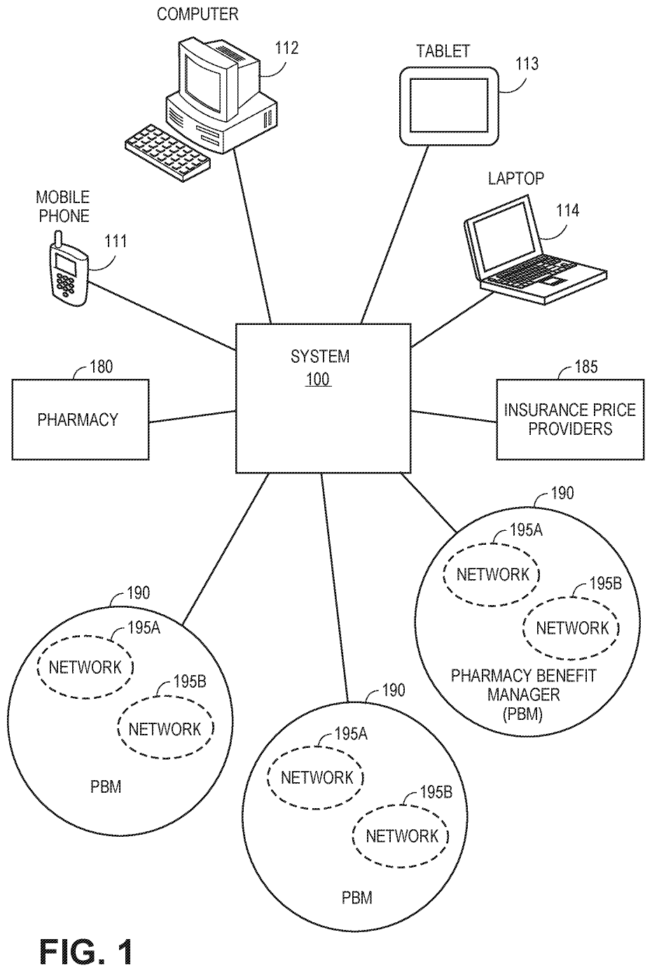

FIG. 1 illustrates an overview of an example system 100 for providing drug prices, according to one aspect of the disclosure. The system 100 may be configured to provide drug pricing information from multiple PBMs 190. The system 100 may communicate with multiple PBMs 190, for example, in order to obtain information relating to prices of various drugs. As explained above, a PBM 190 may administer and/or process claims relating to drugs (e.g., prescription drugs). A PBM 190 may negotiate with one or more pharmacies 180 regarding prices for various drugs (e.g., under a contract or agreement with a pharmacy). For example, PBM 1 and Pharmacy A can form an agreement on how much PBM 1 will compensate Pharmacy A for certain drugs and/or how much Pharmacy A will charge customers for certain drugs.

The pharmacy 180 generally contracts with multiple PBMs 190 for prices for various drugs. The pharmacy 180 may be a part of a pharmacy chain that owns or is associated with multiple locations. For example, large pharmacy chains like CVS, Walgreens, Rite-Aid, etc. have multiple retail locations across the U.S. In such case, the pharmacy chain generally contracts with the PBMs 190 for drug prices, and the retail locations of the pharmacy chain use the prices under the contract.

A PBM 190 generally administers and processes claims associated with a health insurance plan, such as Anthem, Aetna, etc. as part of an insurance network 185 administered by the PBM 190. A “network” may refer to one particular set of prices. For example, a PBM 190 and a pharmacy 180 determine under an agreement what the price for a particular drug will be for a health plan, thereby creating an insurance network 195B. The members of the health plan are charged a certain price for the drug under the agreement between the PBM 190 and the pharmacy 180. Often, as part of the agreement between the PBM 190 and the pharmacy 180, the PBM 190 may also negotiate drug prices for people who are not members of the health plan as part of an unfunded network 195A administered by the PBM. These customers are unfunded because the health plan is not paying for these customers for their prescriptions. These customers may be referred to as “unfunded group” to facilitate explanation. Prices for unfunded group under agreements between PBMs 190 and pharmacies 180 may be referred to as “unfunded drug prices.”

Although the unfunded group may not get the benefit of the prices available to members of the health plan, the unfunded drug prices may still be much less than drug prices cash customers pay. “Cash customers” may refer to customers who purchase a drug without any discounted pricing (e.g., available through a health plan), and the prices the cash customers pay may be referred to as “cash prices.” The unfunded drug prices may be available through a pharmacy discount card or a discount card. Such discount cards may provide discounts on prescription drugs and/or generic drugs. In certain embodiments, the system 100 can obtain and provide information about unfunded drug pricing under various contracts between PBMs 190 and pharmacies 180.

A PBM 190 can administer one or more unfunded networks 195A and insurance networks 195B within the PBM 190. For example, a pharmacy 180 can have multiple unfunded networks 195A within the PBM 190, and also have insurance networks 195B within the PBM 190. In such case, the pharmacy 180 may have one set of prices with the PBM 190 under one network and another set of prices with the PBM 190 under a different network. The price for the same drug may be different from network to network.

An entity associated with the system 100 may negotiate and enter into an agreement with a PBM 190, which is separate from the agreements between the PBM 190 and pharmacies 180, in order to access prices between the PBM 190 and pharmacies 180. For example, the entity may obtain or determine unfunded drug prices under various agreements between the PBM 190 and the pharmacies 180. In some cases, the unfunded prices available to the entity under the separate agreement with the PBMs 190 may not be the exact unfunded prices agreed upon by PBMs 190 and pharmacies 180, but similar prices. In such case, the system 100 may provide users with unfunded prices that are similar or close to the unfunded prices agreed upon between the PBMs 190 and the pharmacies.

The system 100 may also communicate with the pharmacies 180. For example, the system 100 can obtain drug pricing information from a particular pharmacy 180. In some embodiments, the system 100 processes prescription drug claims and may communicate with a pharmacy's system. In certain embodiments, some of the functions provided by the system 100 are integrated into an electronic medical record (EMR) system, and the system 100 may communicate with the EMR system.

The system 100 may also communicate with insurance networks 190B within PBMs to obtain drug pricing information that is associated with a user's health plan when the user provides insurance information. The system 100 may also communicate with other insurance price providers 185 to obtain drug pricing information that is associated with a user's health plan when the user provides insurance information. Any provider of drug pricing information associated with a user's health insurance plan is referred to as the insurance price provider. In some instances, the insurance price provider may be the health plan or another third party contracted with the insurance price provider.

In another embodiment, the user may present the pharmacy 180 with information associated with the system 100 when filling a prescription. For example, the information may be presented on a discount card. The information may include user identification. The information may include a bank identification number (BIN) associated specifically with the system 100. Typically, the BIN routes electronic pharmacy transaction in the direction of the insurance price provider 185. The information may also include a unique processor control number (PCN) associated with the system 100. Typically, the PCN is a secondary identifier that may be used in routing pharmacy transactions to differentiate between health plans provided by the insurance price provider 185. The information may also include a specific group number and member identification number assigned to the user by the system 100 in order to identify the user within the system 100. The pharmacy 180 enters the information into the pharmacy system and the pharmacy transaction is routed to the system 100. Upon receipt of the information, the system 100 retrieves the user profile associated with the user and returns via the pharmacy system, the drug pricing information according to the preferences stored in the user profile. Upon receipt of the information, the system 100 retrieves prices for the prescription at the pharmacy from insurance networks 195B and unfunded networks 195A at several PBMs 190 and/or insurance price providers 185 and sends the prescription to the insurance networks 195B and unfunded networks 195A at several PBMs 190 and/or insurance price providers 185 for processing. The system may also communicate with the user to confirm the user preferences prior to, simultaneously with and/or after returning drug pricing information to the pharmacy 180. The system 100 stores updates to the user preferences in the user profile.

The system 100 can provide a user interface to receive input from a user and/or to display information to the user. For example, the user can enter information for obtaining drug pricing information through the user interface. The user may also enter insurance information through the user interface. The user interface may be provided on various computing devices, such as a mobile phone or a smart phone 111, a computer 112, a tablet 113, a laptop 114, etc.

The user interface can display a portion of the drug pricing information from the PBMs 190, and/or other information to the user. For example, the user interface can display a set of unique identifiers, including a BIN specific to the system. In addition, the user interface can display the price of the drug under the user's health plan when the user provides insurance information. In some instances, the drug price obtained from the insurance network 195B or insurance price provider 185 may be greater than the unfunded prices from the unfunded networks 195A agreed upon between the PBMs 190 and the pharmacies 180. The user interface may query the user via the user interface whether to use insurance or an unfunded rate, receive user input as to whether the user prefers to use the insurance drug pricing or an unfunded rate, and store the user preference in a user profile associated with the user. In an embodiment, a user having a membership profile with insurance information stored in the system 100 may log in to the system 100 to view prices with insurance, instead of re-entering the insurance information each time the system 100 is visited.

The system 100 may also communicate with the user by sending a digital alert to one or more of the various computing devices 111, 112, 113, 114. For example, the digital alert may be one or more of a short message service (SMS) message, a text message, an email, a push notification, a tweet, a communication via social media, etc. In an embodiment, the system 100 sends the digital alert to confirm user preferences. The system 100 may also communicate with the user by sending information to the user through direct mail.

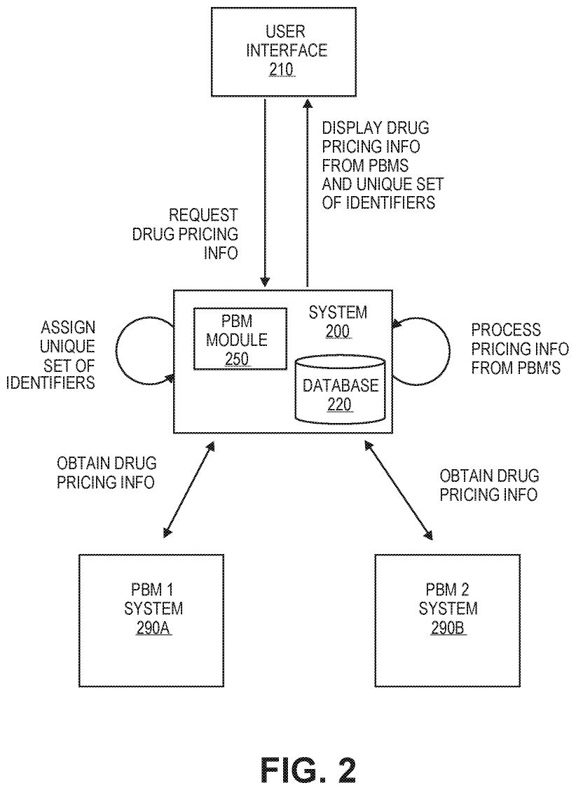

FIG. 2 illustrates an example data flow diagram for providing drug prices from multiple PBMs, according to one aspect of the disclosure. FIG. 2 illustrates a system 200 that can be configured to provide drug prices from multiple PBMs. In an embodiment, the drug pricing described in relation to FIG. 2 are unfunded rates or cash rates. The system 200 may be similar to the system 100 in FIG. 1.

The system 200 can communicate with the systems of PBMs. For example, the system 200 communicates with the PBM 1 system 290A and the PBM 2 system 290B. Such communication may occur through an interface such as an API. The system 200 can include one or more components that may be configured to provide drug prices from multiple PBMs. For example, in FIG. 2, the system 200 includes a PBM Module 250 that can perform or execute functions relating to providing drug prices from multiple PBMs. The system 200 may include one or more other components as appropriate in order to implement the functionality of providing drug prices from multiple PBMs. The system 200 may also include one or more databases 220 to store the drug pricing information. Any component and/or module of the system 200 can reside on one computing device or on separate computing devices.

FIG. 2 illustrates several data flow steps. Data flow steps can occur or be performed in any order. One or more data flow steps may be omitted depending on the embodiment, and additional data flow steps can be added depending on the embodiment. All embodiments described in this disclosure may be implemented separately, together, or in combination. For example, one embodiment may include certain features of another embodiment. In addition, certain features discussed with respect to a particular embodiment may be omitted, and an embodiment may include additional features.

The system 200 obtains drug pricing information from a PBM system 290A, 290B. As explained above, a PBM can negotiate and contract with one or more pharmacies on prices for various drugs. For example, PBM 1 and Pharmacy A agree that the price for Drug A is $20 and Drug B is $30. In some embodiments, a PBM administers multiple networks, and a pharmacy may have different price arrangements with the PBM under each network. For instance, PBM 1 administers claims for Network 1 and Network 2, and the price for Drug A for Pharmacy A under Network 1 is $20 and the price for Drug A for Pharmacy A under Network 2 is $15.

The system 200 can obtain information relating to negotiated drug prices between a PBM and one or more pharmacies from the PBM system 290A, 290B. These prices can be prices for purchase of various drugs at one or more pharmacies. The system 200 may obtain the drug pricing information through an API. The PBM system 290A, 290B may provide an API, which includes functions that can be called by the system 200. The system 200 can call various functions to obtain relevant information. The system 200 may be able to perform mock adjudication of claims using the API in order to figure out drug prices charged by particular pharmacies. A mock adjudication can be performed by submitting information relating to drug name, form, dosage, quantity, pharmacy, etc. The National Drug Code (NDC) may be submitted for mock adjudication. The NDC can refer to a code that is used to identify a drug based on manufacturer, strength, package size, etc. The system 200 may try to determine the NDC for a drug at a particular pharmacy or pharmacy chain to submit for mock adjudication. In one embodiment, the system 200 submits the NDC of the drug, the quantity of the drug, and pharmacy information to the mock adjudication API, and the API returns the price for the drug.

In some cases, the system 200 may have access to actual claims data and determine the prices by analyzing the claims data. By analyzing the claims data, the system 200 can determine how much a pharmacy generally charges for a certain drug, form, dosage, quantity, with a particular PBM.

In some embodiments, the system 200 may not obtain drug pricing information from a PBM, but estimate or calculate the drug prices for a particular pharmacy with that PBM. In one example, a PBM provides a set of pricing rules for determining the price of various drugs. The pricing rules may be based on Average Wholesale Price (AWP). For instance, pricing rules may state that the price of a brand name drug is AWP—20%+dispensing fee and the price of a generic drug is AWP—70%+dispensing fee. Dispensing fee may refer to a fee associated with providing a drug (e.g., filling a prescription). The AWP data can be published by and available from third parties. The PBM may also provide a list of prices called the Maximum Allowable Cost (MAC) list. The MAC list can indicate maximum amounts the PBM pays for brand name drugs and generic drugs, and the prices in the MAC list may be exceptions to the pricing rules. The system 200 can calculate the price of a drug according to the pricing rules, compare it to the price in the MAC list, and provide lower of the two prices. Pricing rules may vary by pharmacy. If a pharmacy has more than one network with a PBM, the pricing rules can differ for each network with the PBM.

In certain embodiments, the system 200 can receive drug prices from sources other than PBMs. In one example, the system 200 receives the information directly from a source, such as a pharmacy. For instance, a pharmacy chain provides a list of the drug prices to the system 200.

In one embodiment, the system 200 obtains usual and customary (U&C) prices for drugs from various sources. A U&C price may refer to a cash price for a drug at a pharmacy. The system 200 can provide U&C prices for a drug, for example, when prices for the drug under agreements between the PBMs and pharmacies are not available. For instance, if a pharmacy does not have an unfunded price for a drug under an agreement with a PBM, the system 200 can display the pharmacy's U&C price for the drug instead.

The system 200 may obtain drug pricing information using patient assistance discounts, which may also be known as co-pay cards. The system 200 may obtain discount drug pricing for the drug by applying the patient assistant discount to the drug price. In certain embodiments, the system 200 obtains patient assistance discounts for drugs from various sources. The system 200 may collect information concerning patient assistance discounts for brand drugs. Patient assistance discounts may include any program or discounts provided by a manufacturer or third party to offer discounts on brand drugs. Such patient assistance discounts may be processed by third party claims processors, such as program administrators, PBMs or other entities. In some embodiments, patient assistance discounts can only be utilized when a user meets eligibility criteria. Such eligibility criteria may include requiring users to have commercial insurance, or to not be eligible for government-provided insurance, or to be of a certain age, or to verify certain personal details. In some embodiments, such patient assistance discounts must be used at a pharmacy along with insurance coverage and may only be applied after a person's insurance information is entered. In other embodiments, such patient assistance discounts may be applied for a user without insurance. The system 200 may provide information concerning patient assistance discounts for the user's drug. For instance, the system 200 can display the patient assistance discount for the drug in addition to an unfunded rate. The system 200 may provide information that allows the patient assistance discount to be processed at the pharmacy for eligible users, such as a BIN, PCN, group number and member ID associated with the patient assistance discount.

The system 200 processes pricing information from the PBMs. In some embodiments, the system 200 obtains the information from the PBM systems 290A, 290B through an API, but the process of requesting and receiving the information may not be fast enough for real-time access. In such case, the system 200 may cache the drug pricing information, e.g., in the database 220. In one example, the mock adjudication results from the PBM API are stored in the database 220. The information processed by the system 200 can be stored in the database 220. FIG. 2 shows one database for illustrative purposes, but the system 200 can include two or more databases.

In some embodiments, the pricing information obtained or determined may be stored in the database 220 prior to or without any processing by the system 200. For example, the system 200 may store any raw data before formatting or transforming the data according to the requirements of the system 200. In other embodiments, the system 200 may not perform any further processing with respect to the information obtained.

The system 200 receives a request for drug pricing information from the user interface 210. The user interface 210 can be provided on a computing device or a display associated with the computing device. The computing device can be, for example, the mobile phone 111, the computer 112, the tablet 113, the laptop 114, etc. as shown in FIG. 1. The user may enter information relating to a drug (e.g., drug name) in the user interface 210, and the associated computing device can generate and send a request for drug pricing information to the system 200. The user interface 210 may be a web interface, a mobile application, or any other appropriate form of user interface.

The request can include any information identifying a drug that the user is interested in finding out prices for. For example, the request can include the name of a drug, and the user enters the name of the drug in the user interface 210. In one embodiment, the user clicks on an advertisement for a particular drug that links to the user interface 210, and the user interface 210 provides the name of the drug without the user having to enter the name. The request may also include location information. Some examples of location information include a zip code, city, address, etc. The location information can be information that the system 200 can use to narrow down the prices to those that are more relevant to a specific geographical area. The user may enter the location information in the user interface 210. In some embodiments, the location information can be determined based on other factors, such as an IP address, with or without user input.

The system 200 can search for or determine one or more prices for the requested drug. The prices may be prices from one or more pharmacies provided under agreements with different PBMs as obtained, determined and/or processed as described above. In some embodiments, the system 200 refers to the drug pricing information in the database 220 to provide relevant prices to the user. The drug pricing information may have been obtained through APIs provided by the PBMs and stored in the database 220. The drug pricing information could also have been extracted from analyzing actual claims data. Or the drug pricing information may have been calculated from pricing rules or estimated in other ways. In other embodiments, the system 200 obtains or determines the prices when a request from the user interface 210 is received. For instance, the prices are calculated according to the pricing rules when the request is received. Various methods of obtaining and/or determining prices, including the methods mentioned above, can be used together, separately, or in some combination. Information relating to prices or used in determining prices may be obtained or updated on demand (e.g., in real-time), periodically (e.g., on a regular basis, at a fixed interval, at a variable interval, etc.), etc. For example, the system 200 can obtain drug pricing information through the API, e.g., on a daily basis or as requested. In another example, the system 200 can obtain the AWP from a third party source, e.g., on a weekly basis. The system 200 may also receive new pricing rules or updates to pricing rules periodically (e.g., every month).

In one embodiment, requests for drug pricing information are received and stored in a queue. Some drug prices are obtained through the API from the PBM, but the speed of API may not be fast enough to provide the drug prices in real-time. In such case, the drug prices are cached. The first time a request is received for a specific drug, for example, for that day, the prices are obtained from the API and stored in the cache. For subsequent requests, the prices are provided from the cache.

In an embodiment, the system 200 can generate and assign a unique set of identifiers to the request for pricing information, which users can utilize to purchase the prescription at or close to the displayed prices at the displayed pharmacies. The unique set of identifiers is displayed to the user via the user interface 210. The unique set of identifiers, which may be presented in the form of a discount card, and include a BIN specific to the system 200, as well as a PCN, group number and member ID. A user may create a user profile that stores the user's information using the user interface 210. When a user profile is created, the unique set of identifiers is assigned and saved to the user profile. The user can then view the unique set of identifiers when logged into the user profile via the user interface 210. In a case where a user profile is not created or a user is not logged into the user profile, a unique set of identifiers may be generated with each pricing request and displayed to the user via the user interface 210.

The system 200 displays a portion of the drug pricing information from PBMs in the user interface 210. Further, the system displays the unique set of identifiers in the user interface. The unique set of identifiers may comprise one or more unique identifiers.

The system 200 can determine prices from one or more pharmacies with multiple PBMs. As explained above, in general, a pharmacy contracts with multiple PBMs, and PBMs contract with multiple pharmacies for drug prices. Therefore, a pharmacy or a pharmacy chain may have multiple prices available for the same drug. In one embodiment, the system 200 displays a portion of the prices from one or more pharmacies, displays a portion of the prices for each pharmacy, and displays the assigned set of identifiers. The set of identifiers will allow the user to receive the prices displayed at the pharmacies displayed.

In other embodiments, the system 200 displays prices for a portion of the pharmacies, displays one price for each pharmacy, and displays the assigned set of identifiers. For instance, the system 200 displays the lowest price for each of the portion of pharmacies displayed. In one example, Pharmacy A has a price for Drug A with PBM 1 and another price for Drug A with PBM 2. Supposing the price for Drug A with PBM 1 is $25 and the price for Drug A with PBM 2 is $15, the system 200 would display the price with PBM 2 since it is lower. Displaying the lowest price available from a pharmacy from multiple PBMs can be beneficial to the users since savings can be maximized. If there are two or more same lowest prices, the system 200 may select one to display arbitrarily or based on a variety of factors. The displayed set of identifiers will allow the user to receive the displayed price at the displayed pharmacy.

The prices displayed in the user interface 210 may be the prices for the most commonly purchased package of the drug. A drug can be manufactured and packaged in different ways. For instance, the same drug can be provided in different form (e.g., tablet, capsule, etc.), dosage (e.g., 10 mg, 20 mg, 40 mg), quantity (e.g., 10, 20, 50, etc.), etc. Form may refer to how the drug is delivered. Some examples of form include tablet, capsule, solution, tube, pump, etc. Dosage may refer to the amount of drug included in each unit or package and can indicate the strength of the drug. The amount can be indicated by weight (e.g., milligram (mg), gram (g), etc.), volume (e.g., milliliter (ml), etc.), etc. Quantity may refer to the number of units included in a package. The same drug may also be available as the brand version or generic version (the version). The user interface 210 may display various options for indicating the package of the drug, and the user can select options corresponding to the package of the drug that the user wants. Such options can include, but is not limited to: generic vs. brand, form, dosage, quantity, etc. The system 200 can update the results to provide prices for the package of the drug selected by the user. The system 200 may also display other information relating to the drug in the user interface 210.

The system 200 can filter or narrow down the prices to be displayed based on the location information. In one embodiment, the results can be filtered based on the zip code entered by the user. Zip code is used as one example, but any other geographical or location indicator can be used, such as an address. The system 200 displays prices associated with pharmacy locations in proximity to the zip code. For example, proximity is determined to be within a default radius from the zip code. The user can select or adjust the radius for the pharmacy locations, and the results are updated accordingly. For example, the user can select 5, 10, 15, 20, 25 miles, etc. from the zip code. If a pharmacy has more than one location within the radius, the user interface 210 may display one price for the pharmacy, and the user can click on the price or another link to view information relating to the different locations. In one embodiment, the user interface 210 displays the addresses for the different locations.

In a certain embodiment, the user is not initially requested to enter location information. The system 200 displays prices for a drug at a portion of the major pharmacy chains without filtering the prices for a geographical area. Then, the user may enter the location information, and the user interface 210 displays the prices relating to the location. In an embodiment, the system 200 displays prices from all of the major pharmacy chains.

In some embodiments, the default or initial radius of pharmacy locations is determined by the system 200 based on factors like population density. For example, in highly populated cities like New York or Los Angeles, a smaller initial radius may be selected, for example, to avoid including too many results. The user can adjust the radius as appropriate in order to view prices for a larger area or a smaller area.

In certain embodiments, the system 200 displays a map of pharmacy locations within the default radius or user designated radius from the location information, along with the prices associated with these pharmacy locations. The map can initially display pharmacy locations in the default radius, and as the user changes the radius, the map can be updated to show pharmacy locations in the changed radius. For instance, if the radius is increased, the map zooms out and displays more locations, and if the radius is decreased, the map zooms in and displays fewer locations.

The system 200 can create an index to reduce the amount of time for searching for prices that are relevant to a specific location or geographical area. In one embodiment, the system 200 creates a two-dimensional (“2D”) geospatial index. For example, when using a 2D geospatial index, the prices are stored with relevant 2D geospatial point(s), and the prices can be queried using the 2D geospatial index. In another embodiment, the system 200 creates a three-dimensional (“3D”) geospatial index. A 3D geospatial index can have the spatial coordinates as the x-axis and the y-axis, and have the drug price as the z-axis.

The system 200 can also filter the prices to be displayed in the user interface 210 based on a variety of factors other than or including the location information. In one embodiment, the user interface 210 displays options relating to pharmacies and the user can choose which types of pharmacies to view prices for. Some examples of pharmacy options or types include: 24-hour, mail order, home delivery, compounding, walk-in clinic, drive-up window, languages spoken, major chains, etc. The system 200 displays a portion of the pharmacies that satisfy the selected user options. In an embodiment, the system 200 displays all of pharmacies that satisfy the selected user options.

In some embodiments, the prices displayed are unfunded prices available under agreements between PBMs and pharmacies for unfunded group. The system 200 provides a discount in order to allow unfunded group to purchase the drug at unfunded prices. In addition, the discounts allow customers to purchase at lower prices among the unfunded drug prices available from multiple PBMs.

The system 200 may rank the drug prices from multiple PBMs prior to displaying drug prices in the user interface 210. The system 200 may display only some of the prices from the ranking process. Generally, if a pharmacy has multiple prices for the same drug under agreements with different PBMs, the system 200 displays one price for the pharmacy. In one embodiment, the system 200 ranks the prices for a pharmacy from lowest to highest and displays the lowest price. For example, a lower price is ranked higher than a higher price. In other embodiments, the system 200 ranks the prices for a pharmacy using methods other than lowest to highest. The system 200 may choose to display only one price from a ranking process in the user interface 210 (e.g., highest ranked or lowest ranked price from the ranking process, etc.).

In one embodiment, the system 200 may rank a higher price from one PBM as higher than, or before, a lower price from another PBM. The system 200 may do so based on various factors. One such factor can be acceptance rate of a price or prices from a PBM at pharmacies. Another factor can be a partnership with a particular PBM. For instance, a PBM may provide more accurate information regarding prices or provide additional benefits that can be passed on to consumers. The system 200 may rank a higher price higher than a lower price if the difference between the higher price and the lower price does not exceed a threshold value (or is less than the threshold value). Ranking a price higher than another price can refer to placing the price before the other price in a sequence. The threshold can be set low such that the impact on the price to the user is minimal. For example, the threshold can be set to $0.10, $0.20, etc. In general, the threshold value is less than $1. The same threshold value may be set for all drugs provided by a PBM, or a different threshold value can be set for a group of drugs or an individual drug provided by a PBM.

In certain embodiments, if there are more than two PBMs, the system 200 can list the PBMs in order of preference and determine multiple threshold values for listing one PBM over another PBM. For instance, there are three PBMs (PBM 1, PBM 2, PBM 3), and the preference for listing prices from these PBMs are in the order of PBM 3, PBM 1, and PBM 2. The system 200 defines a threshold value for listing prices from PBM 3 over prices from PBM 1 and a threshold value for listing prices from PBM 1 over prices from PBM 2. Accordingly, the system 200 can rank prices from multiple PBMs for a pharmacy, given that the price difference between one PBM and another PBM does not exceed the designated threshold.

In one embodiment, the system 200 may determine whether to rank a price from one PBM higher than a price from another PBM based on the percentage of prices to show for a specific PBM. Referring to the example above, if there are three PBMs (PBM 1, PBM 2, PBM 3), the system 200 designates a percentage of prices to display from PBM 1, a percentage of prices to display from PBM 2, and a percentage of prices to display from PBM 3. For instance, the percentage for PBM 3 can be 50%, the percentage for PBM 1 can be 30%, and the percentage for PBM 2 can be 20%. In certain embodiments, the percentage for a PBM is linked to the threshold value for listing one PBM over another. For example, the percentage for displaying prices from PBM 1 is linked to the threshold value for listing PBM 3 over PBM 1 such that when the threshold value is changed, the percentage is updated. If the percentage is changed, the threshold value is updated.

The system 200 can also choose to rank the prices from different PBMs based on various factors other than or in addition to acceptance rate and PBM partnership, such as compensation by a PBM to the entity for a purchase of a drug (e.g., filling a prescription). Such ranking may sort the prices in an order that is different from a lowest-to-highest ranking.

In this manner, the system 200 can allow users to purchase drugs at prices that were not previously available to them by utilizing the set of unique identifiers, which allow users to access the displayed prices at the displayed pharmacies. The entity associated with system 200 can enter into agreements with the PBMs and pass on the savings to users. By obtaining and/or determining drug pricing information from multiple PBMs and selecting lower prices available from each PBM to provide to the users, the entity can create a new discount network of prices. The entity can also make unfunded drug prices available to the users, which can save a significant amount of money. Often, the cash price of a drug is multiple times the unfunded price of the drug. Users without health insurance plans can benefit from reduced prices almost all of the time. And even users with health insurance plans can benefit when a drug is not covered under their plans, requires prior authorization or the system 200 provides better prices for the drug. As explained above, the system 200 can provide a convenient and easy-to-use user interface 210, making it simple for users to find the information they are looking for.

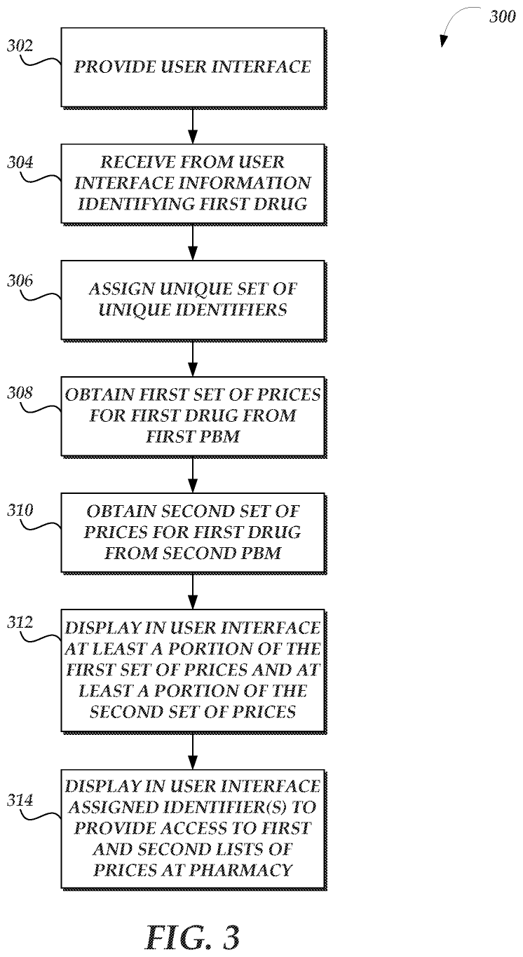

FIG. 3 illustrates one embodiment of an example process 300 for displaying drug prices from multiple for PBMs. The process 300 is described with respect to the system 200 of FIG. 2. However, one or more of the steps of process 300 may be implemented by other systems, such as the system 100 described in FIG. 1. The process 300 can be implemented by any one of, or a combination of, the components of the system 200 (e.g., the PBM module 250). Further details regarding certain aspects of at least some of steps of the process 300 are described in greater detail above with reference to FIGS. 1 and 2.

At block 302, the system 200 provides a user interface 210 for providing drug pricing information. Users can request pricing information about a specific drug via the user interface 210. For example, the user can enter the name of the drug and the user's zip code in the user interface 210, and send a request for pricing information.

At block 304, the system 200 receives from the user interface 210 information identifying a first drug. The information can include the name of the drug. The system 200 can also receive location information from the user interface 210. In an embodiment, the system 200 also receives information identifying the user, such as user name, mailing address, email address, password, etc.

At block 306, the system 200 associates a set of unique identifiers to the request for drug pricing information. In an embodiment, the set of unique identifiers is assigned to specific drug pricing search. In another embodiment, the set of unique identifiers is associated with the user. In a further embodiment, the set of unique identifiers is assigned to the user. The system 200 may correlate the set of unique identifiers with the information identifying the drug and the information identifying the user. The set of unique identifiers may include a BIN assigned to the system 200. In an embodiment, the unique identifier may have a first part comprising a BIN assigned to the system 200 and a second part comprising of identifiers that uniquely identify the user. The set of unique identifiers may include one or more of a Group Number and a Member ID.

At block 308, the system 200 obtains a first set of prices for the first drug that is associated with a first PBM. A PBM may process a claim relating to a drug and have an agreement with a pharmacy relating to a price of one or more drugs (e.g., by a contract). The first set of prices can include at least one price for the first drug, and each price in the first set of prices can be determined by an agreement between the first PBM and a pharmacy. Each price in the first set of prices may be a price for purchase of the first drug at the pharmacy associated with the price.

In one embodiment, at least one price in the first set of prices is obtained by performing of a mock adjudication of a claim relating to the first drug using an API provided by the first PBM. In another embodiment, the at least one price in the first set of prices is obtained based on pricing rules for the first drug provided by the first PBM.

At block 310, the system 200 obtains a second set of prices for the first drug that is associated with a second PBM. The second set of prices can include at least one price for the first drug, and each price in the second set of prices can be determined by an agreement between the second PBM and a pharmacy. Each price in the second set of prices may be a price for purchase of the first drug at the pharmacy associated with the price. The pharmacy for the first set of the prices and the second set of prices may be the same pharmacy or different pharmacies.

In one embodiment, similar to block 308, the at least one price in the second set of prices is obtained by performing of a mock adjudication of a claim relating to the first drug using an API provided by the second PBM. In another embodiment, the at least one price in the second set of prices is obtained based on pricing rules for the first drug provided by the second PBM.

In some embodiments, to obtain drug pricing, the first PBM may administer claims relating to a health insurance plan, and an agreement between the first PBM and a pharmacy may specify a price of the first drug for a member of the health insurance plan and a price of the first drug for a customer that is not a member of the health insurance plan. The first set of prices can include the price of the first drug for a customer that is not a member of the health insurance plan, which may be referred to as the “non-member price.”

Similarly, in some embodiments, to obtain drug pricing, the second PBM may administer claims relating to a health insurance plan. The health insurance plan may be the same plan as or a different plan from the one administered by the first PBM. An agreement between the second PBM and a pharmacy may specify a price of the first drug for a member of the health insurance plan and a price of the first drug for a customer that is not a member of the health insurance plan. The second set of prices can include the price of the first drug for a customer that is not a member of the health insurance plan. The pharmacy may be the same pharmacy with which the first PBM has an agreement or a different pharmacy from the pharmacy with which the first PBM has an agreement. In these embodiments, the system 200 may display the non-member price for the first drug included in the first set of prices, or the non-member price for the first drug included in the second set of prices.

At block 312, the system 200 displays in the user interface 210 at least a portion of the first set of prices and the second set of prices. The first set of prices and the second set of prices each include at least one price for the same drug, but the system 200 may not list a price from each set. For example, if the price of the first drug in the first set and the price of the first drug in the second set are for the same pharmacy, the system 200 can list one of the prices (e.g., the lower price).

In one embodiment, the first set of prices includes a price for the first drug determined by an agreement between the first PBM and a pharmacy, and the second set of prices includes a second price for the first drug determined by an agreement between the second PBM and the pharmacy. The system 200 compares the first price for the first drug and the second price for the first drug, and displays lower of the first price for the first drug and the second price for the first drug.

In some embodiments, the system 200 filters the first set of prices and the second set of prices based on the location information prior to displaying at least a portion of the first set of prices and the second set of prices in the user interface 210.

At block 314, the system displays the unique set of identifiers, including a BIN specific to the system 200, in the user interface. The user may present the unique set of identifiers at pharmacies in order to receive the prices displayed.

The process 300 can include fewer, more, or different blocks than those illustrated in FIG. 3 without departing from the spirit and scope of the description. Moreover, it will be appreciated by those skilled in the art and others that some or all of the functions described in this disclosure may be embodied in software executed by one or more processors of the disclosed components and mobile communication devices. The software may be persistently stored in any type of non-volatile storage.

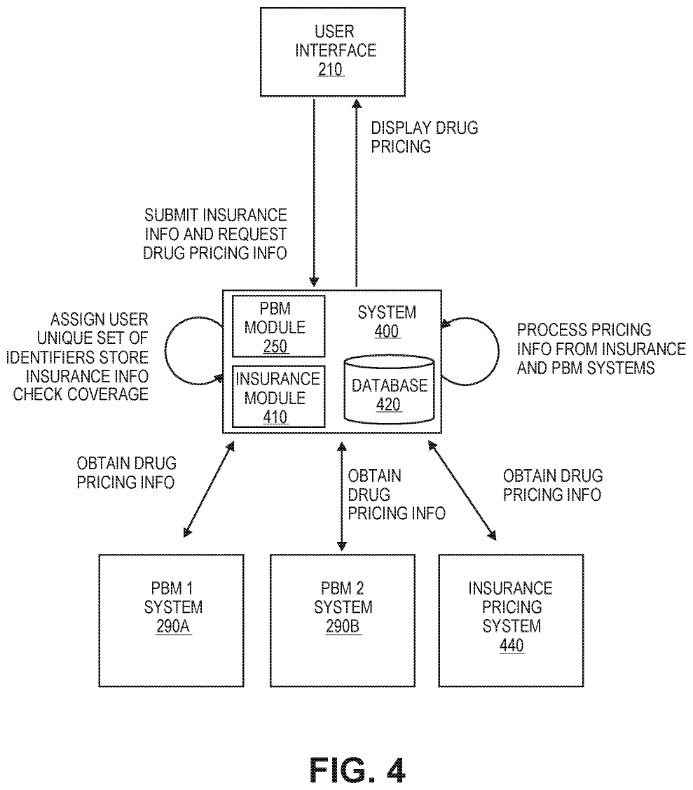

FIG. 4 illustrates an example data flow diagram for providing drug prices from multiple PBMs and the insurance price associated with the user health plan from insurance price providers. FIG. 4 illustrates a system 400 that can be configured to provide drug prices from multiple PBMs and from insurance price providers. The system 400 may be configured to provide drug prices from multiple PBMs periodically. The system 400 may be similar to the system 100 in FIG. 1 and the system 200 in FIG. 2.

The system 400 can communicate with the systems of PBMs. For example, the system 400 communicates with the PBM 1 system 290A and the PBM 2 system 290B as described above with respect to FIG. 2. Such communication may occur through an interface, such as an API. The system 400 can include one or more components that may be configured to provide drug prices from multiple PBMs. For example, in FIG. 4, the system 400 includes the PBM Module 250 that can perform or execute functions relating to providing drug prices from multiple PBMs.

The system 400 can communicate with the systems of insurance price providers. For example, the system 400 communicates with the insurance pricing system 440. Although one insurance pricing system 440 is illustrated in FIG. 4, the system 400 can communicate with multiple insurance pricing systems 440. Insurance pricing system 440 represents an entity, such as a PBM, health insurance provider associated with the users' health plan or the users' prescription benefit plan or other third party that provides insurance pricing to the system 400. An insurance pricing system 440 may be the same PBMs used by the system 400 to provide unfunded rates.

Such communication may occur through an interface, such as an API. The system 400 can include one or more components that may be configured to provide drug prices from the multiple insurance price providers as represented by the insurance pricing system 440. For example, in FIG. 4, the system 400 includes an insurance module 410 that can perform or execute functions relating to providing drug prices from insurance networks of PBMs as represented by the insurance pricing system 440. The system 400 may include one or more other components as appropriate in order to implement the functionality of providing drug prices from multiple insurance price providers of the insurance pricing system 440.

The system 400 may also include one or more databases 420 to store the drug pricing information. FIG. 4 shows one database for illustrative purposes, but the system 400 can include two or more databases. Any component and/or module of the system 400 can reside on one computing device or on separate computing devices.

FIG. 4 illustrates several data flow steps. Data flow steps can occur or be performed in any order. One or more data flow steps may be omitted depending on the embodiment, and additional data flow steps can be added depending on the embodiment. All embodiments described in this disclosure may be implemented separately, together, or in combination. For example, one embodiment may include certain features of another embodiment. In addition, certain features discussed with respect to a particular embodiment may be omitted, and an embodiment may include additional features.

The system 400 obtains drug pricing information from a PBM system 290A, 290B, as described above. The system 400 may obtain the drug pricing information through an API. The PBM system 290A, 290B may provide an API, which includes functions that can be called by the system 400. The system 400 can call various functions to obtain relevant information. The information processed by the system 400 from the PBM systems 290A, 290B can be stored in the database 420.

The system 400 obtains drug pricing information from the insurance pricing system 440. The system 400 may obtain the drug pricing information through an API. The insurance pricing system 440 may provide an API, which includes functions that can be called by the system 400. The system 400 can call various functions to obtain relevant information. The information processed by the system 400 from the insurance pricing system 440 can be stored in the database 420.

The system 400 receives a request for drug pricing information from the user interface 210. The user interface 210 can be provided on a computing device or a display associated with the computing device. The computing device can be, for example, the mobile phone 111, the computer 112, the tablet 113, the laptop 114, etc. as shown in FIG. 1. The user may enter information relating to a drug (e.g., drug name) in the user interface 210, and the associated computing device can generate and send a request for drug pricing information to the system 400. The request may include location information as described above with respect to FIG. 2. The user interface 210 may be a web interface, a mobile application, or any other appropriate form of user interface.

The request can include any information identifying a drug that the user is interested in obtaining pricing information. For example, the request can include the name of a drug, and the user enters the name of the drug in the user interface 210. The request may also include location information. Some examples of location information include a zip code, city, address, etc. The location information can be information that the system 400 can use to narrow down the prices to those that are more relevant to a specific geographical area. The user may enter the location information in the user interface 210. In some embodiments, the location information can be determined based on other factors, such as an IP address, with or without user input.

The request can also include health insurance information, which the user enters in the user interface 210. For example, the request can include the user's name, insurance provider, group number, plan code, issuer number, member ID, and RX BIN number associated with the user's health insurance plan.

The system 400 can search for or determine one or more prices for the requested drug from the PBM systems 290A, 290B and, if the user's health insurance plan information is provided, from the insurance pricing system 440. The request drug pricing information may be for a prescription. The system 400 may search for or determine one or more prices for a generic equivalent or alternative therapeutic. The system 400 may search for or determine one or more prices for different dosages or forms of the prescription.

The system 400 can obtain the drug pricing information acquired from the PBM systems 290A, 290B in one or more of the ways described above with respect to FIG. 2. For example, the prices may be prices from one or more pharmacies provided under agreements with different PBMs as obtained, determined and/or processed as described above. In some embodiments, the system 400 refers to the drug pricing information in the database 420 to provide relevant prices to the user. The drug pricing information may have been obtained through APIs provided by the PBMs and stored in the database 420. For instance, the prices are calculated according to the pricing rules when the request is received. Various methods of obtaining and/or determining prices, including the methods mentioned above, can be used together, separately, or in some combination. In one embodiment, the drug pricing information returned from the PBM systems 290A, 290B are unfunded rates.

The system 400 uses the insurance information to obtain the drug pricing information from the insurance pricing system 440 according to the user's health plan or prescription benefit plan. In an embodiment, the system 400 verifies that the user is covered by the insurance pricing system 440. In an embodiment, the system 400 verifies that the requested drug is covered by the insurance pricing system 440. In an embodiment, the system 440 determines whether additional approvals and/or steps are necessary to obtain the drug and/or drug pricing, such as whether a drug requires prior authorization. In an embodiment, the user creates a user profile via user interface 210 and the system 400 stores the user profile in the database 420 and the insurance information in the database 420 as part of the user profile. In an embodiment, the system 400 may generate a profile for the user without the user entering information, for instance, by generating a profile based on a mobile ID.

The system 400 obtains insurance drug prices from insurance pricing system 440. There are several ways the system 400 may obtain the insurance drug pricing from insurance pricing system 440. For example, the system 400 may be able to perform mock adjudication of claims using an API in order to determine insurance drug prices charged by particular pharmacies. A mock adjudication can be performed by submitting the insurance information and information relating to drug name, form, dosage, quantity, pharmacy, etc. The National Drug Code (NDC) may be submitted for mock adjudication. The NDC can refer to a code that is used to identify a drug based on manufacturer, strength, package size, etc. The system 400 may try to determine the NDC for a drug at a particular pharmacy or pharmacy chain to submit for mock adjudication. In one embodiment, the system 400 submits the insurance information, the NDC of the drug, the quantity of the drug, and pharmacy information to the mock adjudication API, and the API returns the price for the drug.

In some cases, the system 400 may obtain insurance pricing by having access to actual claims data and determine the prices by analyzing the claims data. By analyzing the claims data, the system 400 can determine how much an insurance price may be on a particular plan, at a particular pharmacy and/or for a certain drug, form, dosage, quantity.

In some embodiments, the system 400 may not obtain drug pricing information from an insurance pricing system, but estimate or calculate the drug prices. In one example, the system may possess a set of pricing rules for determining the price of various drugs with an insurance plan. The pricing rules may be based on Average Wholesale Price (AWP). For instance, pricing rules may state that the price of a brand name drug is AWP—20%+dispensing fee and the price of a generic drug is AWP—70%+dispensing fee. Dispensing fee may refer to a fee associated with providing a drug (e.g., filling a prescription). The AWP data can be published by and available from third parties. The system may also have a list of prices called the Maximum Allowable Cost (MAC) list. The MAC list can indicate maximum amounts an insurance plan pays for brand name drugs and generic drugs, and the prices in the MAC list may be exceptions to the pricing rules. The system 400 can calculate the price of a drug according to the pricing rules, compare it to the price in the MAC list, and provide lower of the two prices. Pricing rules may vary by plan and by pharmacy.

In certain embodiments, the system 400 can receive drug prices from other sources. In one example, a user may provide insurance pricing via the user interface 210 and the system 400 may use the provided insurance pricing for other users with the same insurance plan.

In one embodiment, requests for drug pricing information are received and stored in a queue. Some drug prices are obtained through the API from the insurance pricing system 440, but the speed of API may not be fast enough to provide the drug prices in real-time. In such case, the drug prices are cached. The first time a request is received for a specific drug, for example, for that day, the prices are obtained from the API and stored in the cache. For subsequent requests, the prices are provided from the cache. In some embodiments, the pricing information obtained or determined may be stored in the database 420 prior to or without any processing by the system 400. For example, the system 400 may store any raw data before formatting or transforming the data according to the requirements of the system 400. In other embodiments, the system 400 may not perform any further processing with respect to the information obtained.

The system 400 can also choose to rank the prices from different insurance pricing systems 440 based on various factors other than or in addition to acceptance rate and PBM or insurance company partnership, such as compensation to the entity for a purchase of a drug (e.g., filling a prescription). Such ranking may sort the prices in an order that is different from a lowest-to-highest ranking.

In another example, the system 400 may be able to perform an actual adjudication of claims using an API in order to determine insurance drug prices charged by particular pharmacies. In one embodiment, the system 400 submits the insurance information, the NDC of the drug, the quantity of the drug, and pharmacy information to the adjudication API, and the API returns the price for the drug.

In an embodiment, the system 400 assigns a set of unique identifiers to the user and correlates the information identifying the user's insurance, the information identifying the user and the information identifying the drug with the assigned set of unique identifiers to create a user profile. The set of unique identifiers may include a BIN assigned to the system 400. In an embodiment, the unique identifier may have a first part comprising a BIN assigned to the system 400 and a second part comprising of identifiers that uniquely identify the user. The set of unique identifiers may include one or more of a Group Number and a Member ID.

Further, user preferences are associated or correlated with the user profile. Examples of user preferences are always use the insurance to obtain a drug, or always use a lower price to obtain the drug, preferred form of drug (liquid, tablet, chewable, etc.), and the like.

The user profile is stored in the database 420. For example, the user profile may include the assigned set of unique identifiers, user name, contact information (mailing address, email address, mobile number, etc.), preferred way of contacting, insurance information (insurance company, group number, RX BIN, member ID, etc.), drug pricing preferences (health plan pricing through insurance company, lowest price available, lowest price available within a distance from user's location, mail order pricing, and the like) and user's prescriptions.

When the user and drug are covered by insurance, the system 400 displays the insurance drug price from the insurance pricing system and a portion of the drug pricing information from PBMs 290A, 290B in the user interface 210. The user selects in the user interface 210 whether to use the insurance drug pricing or to use the PBM 290A, 290B drug pricing. The system 400 receives from the user interface 210 the user's selection and stores the user's selection as a user preference in the user profile. When the drug is not covered by insurance as indicated by the data returned from insurance pricing system 440, the system 400 displays a portion of the drug pricing information from PBMs 290A, 290B in the user interface 210. Further, the system 400 displays the assigned set of unique identifiers in the user interface 210.

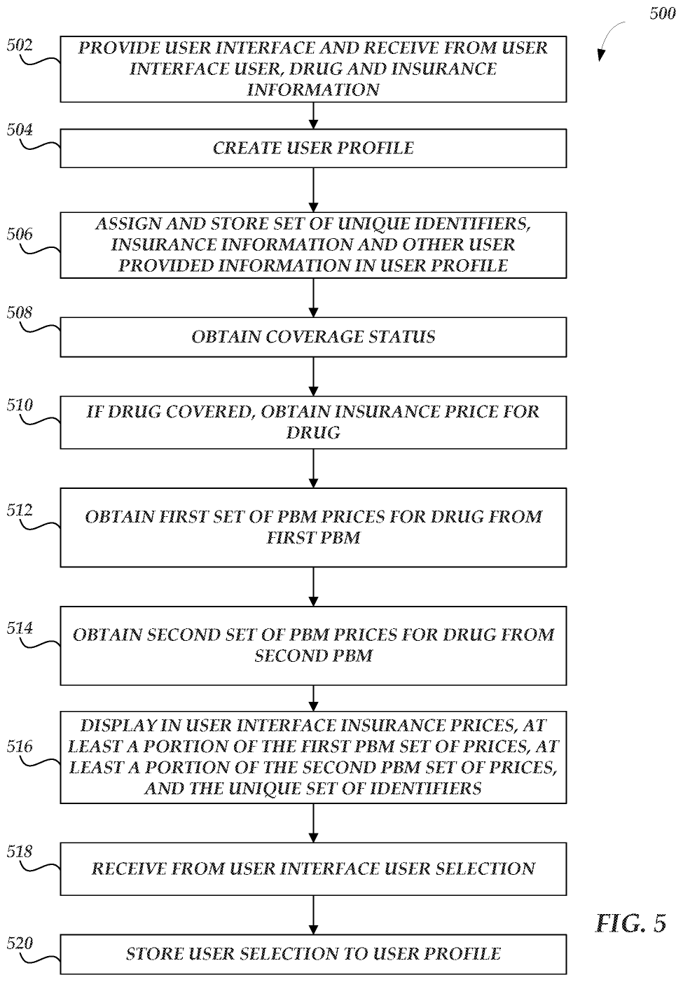

FIG. 5 illustrates one embodiment of an example process 500 for displaying drug prices from the insurance pricing system 440 and multiple PBMs 290A, 290B. The process 500 is described with respect to the system 400 of FIG. 4. However, one or more of the steps of process 500 may be implemented by other systems, such as the system 100 illustrated in FIG. 1 and the system 200 illustrated in FIG. 2. The process 500 can be implemented by any one of, or a combination of, the components of the system 400 (e.g., the PBM module 250 and the insurance module 410). Further details regarding certain aspects of at least some of steps of the process 500 are described in greater detail above with reference to FIGS. 1-4.

At block 502, the system 400 provides a user interface 210 for providing drug pricing information from multiple PBMs 290A, 290B and from the insurance pricing system 440. Users can request pricing information about a specific drug, supply their insurance information, and supply user identifying information (user name, mailing address, email address, password etc.) via the user interface 210. For example, the user can enter a user name, mailing address, and email address, password, the name of the drug, and the insurance information in the user interface 210, and send a request for pricing information to the system 400.

The system 400 receives from the user interface 210 information to create a user profile (user name, password, mailing address, email address, mobile number, etc.). The system 400 also receives from the user interface 210 information identifying a first drug and information identifying the user's insurance. The information identifying the first drug can include the name of the drug. The information identifying the insurance pricing system 440 can include the name of the insurance company, issuer number, group number, plan number, plan name, RX BIN, membership ID, names of the individuals covered under the insurance, etc. The system 400 can also receive location information from the user interface 210.

At block 504, the system 400 creates a user profile associated with the user based at least in part on the user information received from the user interface 210. The user profile is stored in the database 420.

At block 506, the system 400 associates a set of unique identifiers, information identifying a first drug, information identifying the user's insurance, and information identifying the user to the user's profile. The system 400 may correlate the set of unique identifier with the information identifying the drug, the information identifying the insurance pricing system 440, and the information identifying the user. The set of unique identifiers may include a BIN assigned to the system 400. In an embodiment, the unique identifier may have a first part comprising a BIN assigned to the system 400 and a second part comprising of identifiers that uniquely identify the user. The set of unique identifiers may include one or more of a Group Number and a Member ID.

At block 508, the system 400 queries the insurance pricing system 440 using the insurance information provided by the user to determine whether the user is covered under the insurance. In response to the query, the system 400 receives the user's coverage status from the insurance pricing system 440. In an embodiment, the system 400 queries the insurance pricing system 440 using the information identifying the insurance pricing system 440 and the information identifying the first drug that is provided by the user to determine whether the drug is covered under the insurance. In an embodiment, the insurance pricing system 440 is a PBM that provides and administers insurance pricing via its insurance networks. Such communication may occur through an interface, such as an API.

At block 510, when the user and the drug are covered under the insurance, the system 400 obtains the price of the drug through the user's insurance from the insurance pricing system 440. The system 400 may be able to perform mock adjudication or actual adjudication of claims using an API in order to determine insurance drug prices charged by particular pharmacies. A mock adjudication, for example, can be performed by submitting the insurance information and information relating to drug name, form, dosage, quantity, pharmacy, etc. The National Drug Code (NDC) may be submitted for mock adjudication. The NDC can refer to a code that is used to identify a drug based on manufacturer, strength, package size, etc. The system 400 may try to determine the NDC for a drug at a particular pharmacy or pharmacy chain to submit for mock adjudication. In one embodiment, the system 400 submits the insurance information, the NDC of the drug, the quantity of the drug, and pharmacy. Blocks 508 and 510 of the process 500 can be implemented by the insurance module 410.

At block 512, the system 400 obtains a first set of prices for the first drug that is associated with the first PBM 290A as described above in FIG. 3, block 308. A PBM may process a claim relating to a drug and have an agreement with a pharmacy relating to a price of one or more drugs (e.g., by a contract). The first set of prices can include at least one price for the first drug, and each price in the first set of prices can be determined by an agreement between the first PBM and a pharmacy. Each price in the first set of prices may be a price for purchase of the first drug at the pharmacy associated with the price. In an embodiment, the prices are unfunded prices.

In one embodiment, at least one price in the first set of prices is obtained by performing a mock adjudication of a claim relating to the first drug using an API provided by the first PBM 290A. In another embodiment, the at least one price in the first set of prices is obtained based on pricing rules for the first drug provided by the first PBM 290A.

At block 514, the system 400 obtains a second set of prices for the first drug that is associated with the second PBM 290B as described above in FIG. 3, block 310. The second set of prices can include at least one price for the first drug, and each price in the second set of prices can be determined by an agreement between the second PBM and a pharmacy. Each price in the second set of prices may be a price for purchase of the first drug at the pharmacy associated with the price. The pharmacy for the first set of the prices and the second set of prices may be the same pharmacy or different pharmacies. In an embodiment, the prices are unfunded prices.

In one embodiment, similar to block 512, at least one price in the second set of prices is obtained by performing of a mock adjudication of a claim relating to the first drug using an API provided by the second PBM 290B. In another embodiment, the at least one price in the second set of prices is obtained based on pricing rules for the first drug provided by the second PBM 290B.

In some embodiments, the first PBM may administer claims relating to a health insurance plan, and an agreement between the first PBM and a pharmacy may specify a price of the first drug for a member of the health insurance plan and a price of the first drug for a customer that is not a member of the health insurance plan. The first set of prices can include the price of the first drug for a customer that is not a member of the health insurance plan, which may be referred to as the “non-member price.”

Similarly, the second PBM may administer claims relating to a health insurance plan. The health insurance plan may be the same plan as or a different plan from the one administered by the first PBM. An agreement between the second PBM and a pharmacy may specify a price of the first drug for a member of the health insurance plan and a price of the first drug for a customer that is not a member of the health insurance plan. The second set of prices can include the price of the first drug for a customer that is not a member of the health insurance plan. The pharmacy may be the same pharmacy with which the first PBM has an agreement or a different pharmacy from the pharmacy with which the first PBM has an agreement. In these embodiments, the system 400 may display the non-member price for the first drug included in the first set of prices, or the non-member price for the first drug included in the second set of prices.

Blocks 512 and 514 of the process 500 can be implemented by the PBM module 250.

At block 516, the system 400 displays in the user interface 210 the insurance prices of the drug when the drug is covered by the insurance, at least a portion of the first set of prices and the second set of prices from the PBMs, and the set of unique identifiers that users can utilize at the pharmacy to obtain the pricing displayed. The first set of prices and the second set of prices each include at least one price for the same drug, but the system 400 may not list a price from each set. For example, if the price of the first drug in the first set and the price of the first drug in the second set are for the same pharmacy, the system 400 can list one of the prices (e.g., the lower price).

The system 400 may further display a message in the user interface 210. The message may be a reminder or recommendation from the system 400 to the user. Examples of message types and message content are, but not limited to, (1) reminder to pick up a prescription; (2) recommendation to transfer the prescription to another pharmacy; (3) recommendation for a generic alternative; (4) recommendation for an alternative therapeutic; (5) recommendation for a different day supply; (6) recommendation for a different dosage, different form, and/or different quantity, etc.; (7) information concerning the user's insurance deductible; (8) an amount the user has spent toward the deductible; (9) the difference between what has been spent and the user's maximum out of pocket expenses; (10) refill reminder; (11) reminder to obtain a new prescription; (12) recommendation to use patient assistance discount; and the like.

In one embodiment, the first set of prices includes an unfunded price for the first drug determined by an agreement between the first PBM and a pharmacy, and the second set of prices includes a second unfunded price for the first drug determined by an agreement between the second PBM and the pharmacy. The system 400 compares the first price for the first drug and the second price for the first drug, and displays lower of the first price for the first drug and the second price for the first drug.

In some embodiments, the system 400 filters the first set of prices and the second set of prices based on the location information prior to displaying at least a portion of the first set of prices and the second set of prices in the user interface 210.

The user may select in the user interface 210 the insurance pricing or the pricing from the at least a portion of the first set of prices and the second set of prices from the PBMs.

At block 518, the system 400 receives from the user interface 210 the user selected pricing. The user may have selected the insurance pricing for the drug. For example, to reach an out-of-pocket limit for medical care spending set by the insurance plan, the user may desire to use insurance to purchase the drug. The user may have selected the pricing from the display of at least a portion of the first set of prices and the second set of prices from the PBMs because the prices provided by the PBMs may have a lower price than the insurance for the drug.

At block 520, the system 400 stores the user's selected drug pricing in the user profile.

The process 500 can include fewer, more, or different blocks than those illustrated in FIG. 5 without departing from the spirit and scope of the description. Moreover, it will be appreciated by those skilled in the art and others that some or all of the functions described in this disclosure may be embodied in software executed by one or more processors of the disclosed components and mobile communication devices. The software may be persistently stored in any type of non-volatile storage.

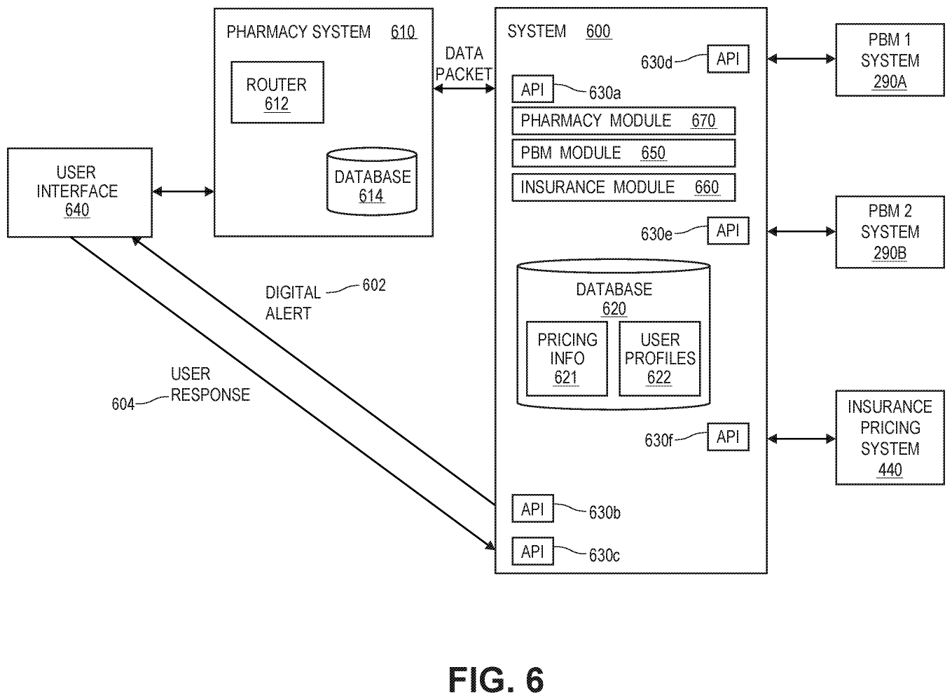

FIG. 6 illustrates an example data flow diagram to provide drug prices in response to a user profile. FIG. 6 illustrates a system 600 that can be configured to receive a message from a pharmacy system 610 comprising a request for drug pricing, obtain drug pricing from multiple PBMs 290A, 290B and insurance drug pricing from an insurance pricing system 440, and return the drug pricing to the pharmacy system 610. The system 600 may also be configured to send digital alerts 602 to a user interface 640 and receive digital responses 604 from the user interface 640. The system 600 may be configured to provide drug prices from multiple PBMs 290A, 290B periodically. The system 600 may have similarities with the system 100 in FIG. 1, the system 200 in FIG. 2, and the system 400 in FIG. 4.

The system 600 can communicate with the systems of PBMs. For example, the system 600 communicates with the PBM 1 system 290A and the PBM 2 system 290B as described above with respect to FIG. 2. Such communication may occur through an interface, such as an API 630d, 630e. The system 600 can include one or more components that may be configured to provide drug prices from multiple PBMs 290A, 290B. For example, in FIG. 6, the system 600 includes the PBM Module 650 that can perform or execute functions relating to providing drug prices from multiple PBMs 290A, 290B.

The system 600 can communicate with the systems of insurance price providers. For example, the system 600 communicates with the insurance pricing system 440 as described above with respect to FIG. 4. Such communication may occur through an interface, such as an API 630f. The system 600 can include one or more components that may be configured to provide drug prices the insurance pricing system 440. For example, in FIG. 6, the system 600 includes the insurance module 660 that can perform or execute functions relating to providing drug prices from the insurance pricing system 440. Although one insurance pricing system 440 is illustrated in FIG. 6, the system 600 can communicate with multiple insurance pricing systems 440. Insurance pricing system 440 represents the insurance price providers associated with the users' health plan or the users' prescription benefit plan. An insurance price provider may be a PBM with an insurance network, a health insurance plan or any entity that provides insurance prices.

The system 600 can communicate with the pharmacy system 610. Such communication may occur through an interface, such as an API 630a. The system 600 can include one or more components that may be configured to receive requests for drug pricing from the pharmacy system 610 and return drug pricing to the pharmacy system 610. For example, in FIG. 6, the system 600 includes a pharmacy module 670 that can perform or execute functions relating to receiving requests for drug pricing and returning drug pricing. In an embodiment, the message format for messages between the system 600 and the pharmacy system 610 are in accordance with the National Council for Prescription Drug Programs (NCPDP) standards. For example, NCPDP Version D.0 defines the data structure and content of single point-of-sale transmissions.

The pharmacy system 610 may include a router 612 and a database 614 to facilitate communications with the system 600. In an embodiment, the messages from the pharmacy system 610 comply with the appropriate NCPDP standard.

The system 600 can communicate with users via a user interface 640. Such communication may occur through interfaces, such as an API 630b used to send digital alerts 602 to the user interface 650 and an API 630c used to receive responses 602 to the digital alerts 602 from the user interface 640. The user interface 640 can be provided on a computing device or a display associated with the computing device. The computing device can be, for example, the mobile phone 111, the computer 112, the tablet 113, the laptop 114, etc. as shown in FIG. 1.

The system 600 may also include one or more databases 620 to store the drug pricing information. FIG. 6 shows one database for illustrative purposes, but the system 600 can include two or more databases. Any component and/or module of the system 600 can reside on one computing device or on separate computing devices. Database 620 may store drug pricing information 621 returned from the multiple PBM systems 290A, 290B, and the insurance pricing system 440. Database 620 may store user profiles 622, which may include membership information, contact preferences, insurance information, drug form preferences (liquid, chewable, etc.) and pricing profiles associated with the users.

FIG. 6 illustrates several data flow steps. Data flow steps can occur or be performed in any order. One or more data flow steps may be omitted depending on the embodiment, and additional data flow steps can be added depending on the embodiment. All embodiments described in this disclosure may be implemented separately, together, or in combination. For example, one embodiment may include certain features of another embodiment. In addition, certain features discussed with respect to a particular embodiment may be omitted, and an embodiment may include additional features.

The system 600 obtains drug pricing information from multiple PBM systems 290A, 290B, as described above. The multiple PBM systems 290A, 290B may each provide an API, which includes functions that can be called by the system 600. The system 600 may obtain the drug pricing information through the APIs 630d, 630e. The system 600 can call various functions to obtain relevant information. The information processed by the system 600 from the PBM systems 290A, 290B can be stored as pricing information 621 in the database 620. The request drug pricing information may be for a prescription. The system 600 may search for or determine one or more prices for a generic equivalent or alternative therapeutic. The system 600 may search for or determine one or more prices for various dosages or forms of a prescription.

The system 600 obtains drug pricing information from the insurance pricing system 440, as described above. The insurance pricing system 440 may provide an API, which includes functions that can be called by the system 600. The system 600 may obtain the drug pricing information through the API 630f. The system 600 can call various functions to obtain relevant information. The information processed by the system 600 from the insurance pricing system 440 can be stored as pricing information 621 in the database 620.

In an embodiment, the user, using the user interface 640, may register for a user profile, which will be stored on the system 600. For example, the user may provide identification information, such as name, mailing address, email address, mobile number, insurance information, such as insurance provider name, group number, member ID, plan name and/or number and user's prescriptions. In addition, the user may provide information that indicates the user's preference in filling a prescription. For example, the user may desire to fill prescriptions through the insurance pricing system 440 associated with the user's health plan. The user may find filling prescriptions through the insurance pricing system 440 advantageous to meet an out-of-pocket yearly deductible. Alternatively, the user may desire to pay the lower cost to obtain the drug. The user may have no preference.

The system 600 receives the registration information and creates a user profile for the user using the enrollment information. In one embodiment, the system 600 may generate a profile without any user registration, such as using the mobile ID of a user who is using the user interface 640 via a mobile application. The system 600 stores the user profile in the database 620 as a user profile 622. The user profile 622 may include the user information, insurance information, and the user drug pricing profile. The user's drug pricing profile may include whether the user prefers to use the insurance or the lower price to fill the prescription.

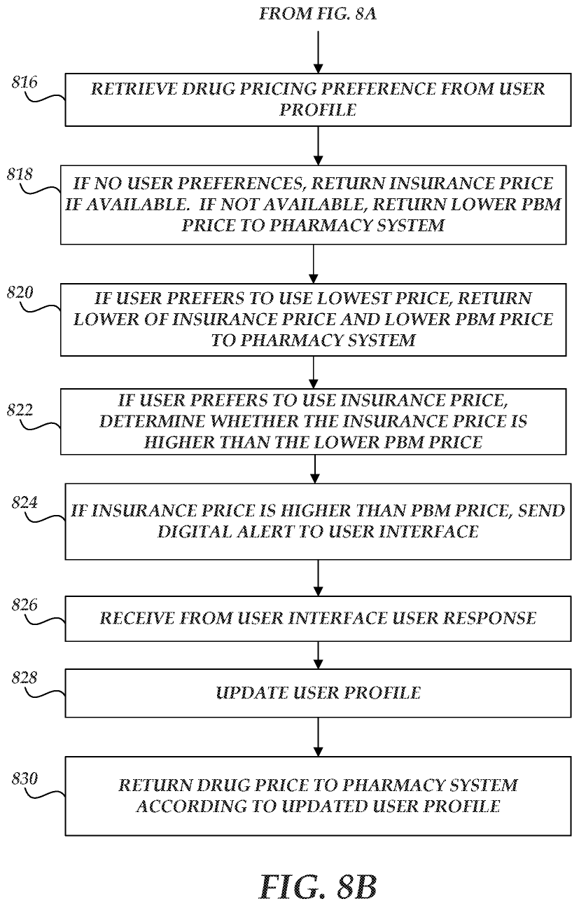

The system 600 associates or correlates a unique set of identifiers with the user profile 622. The system 600 sends a unique set of identifiers to the user in response to receiving the registration information and associates or correlates the unique set of identifiers with the user profile 622 stored in the database 620. The unique set of identifiers may be presented through the user interface 640 and may be in the form of a digital discount card (provided via the website, mobile application, email or text) or physical discount card. An example of a discount card is illustrated in FIG. 9. The discount card may include a Bank Identification Number (BIN) associated with the system 600, a Processor Control Number (PCN) associated with the system 600, a GROUP associated with the system 600, and a member ID associated with the system 600, which is not the same as the membership ID associated with the insurance pricing system 440.

The BIN is a field in the telecommunication standard that is used for the routing and identifying pharmacy claims. The BIN can be used to route electronic transactions from pharmacy systems 610 to the payer of the prescription, such as the PBM that the insurance pricing system 440 associated with the user's health plan has contracted with. BIN's may be a 6 digit number. In some embodiments, the BIN may be referred to as an Issuer Identification Number (IIN) and may be an 8 digit number. In other embodiments, the BIN/IIN may be 7 digits, more than 8 digits, or less 6 digits.

The PCN is a secondary identifier that may be used in routing of pharmacy transactions. The PCN may be defined by the recipient of the electronic transaction routed from the pharmacy system 610. The PCN may be used to differentiate different health plans within the recipient of the electronic transaction, such as different networks 195 within the PBMs 190 illustrated in FIG. 1.

In an embodiment, the unique set of identifiers comprises at least a unique BIN that routes electronic transmissions from the pharmacy system 610 to the system 600. A user presents a prescription to be filled and the unique set of identifiers to the pharmacy system 610. For example, the unique set of identifiers may be presented online via the user interface 640, via email and/or text, or by presenting a physical discount card at the pharmacy counter.

The pharmacy system 610 collects pertinent information, such as name, dosage, form, quantity, number of refills, etc., about the drug from the prescription. The pharmacy system 610 also collects the user's unique set of identifiers assigned by system 600, including at least the BIN associated with the system 600.

The pharmacy system 610 formats a data packet with the collected information to send to the system 600 for drug pricing information, in the same manner that the pharmacy system 600 would format a data packet to send to a PBM for drug pricing information.

An example of the syntax of a transmission request and response may be as follows:

Header Segment

Header field segments

Segment Separator

Required segments within segment, as appropriate, with filed

separators

Optional segment fields with field separators

Segment Separator

Required segments within segment, as appropriate, with filed

separators

Optional segment fields with field separators

Group Separator

Segment Separator

Required segments within segment, as appropriate, with filed

separators

Optional segment fields with field separators

Segment Separator

Required segments within segment, as appropriate, with filed

separators

Optional segment fields with field separators

Table 1

The transmission from the pharmacy system 610 may comprise one or more of the following data fields:

Submitted ID

Fill Date

User Name

BIN

PCN

Group ID

Member ID

Claim Status

Rx Written Date

Posting Date

National Drug Code (NDC) Number

Product Name

Quantity

Days Supply

GCN

Brand/Generic Indicator

NCPDP Number for pharmacy

Pharmacy National Provider Identifier (NPI)

Pharmacy Name

Pharmacy Address 1

Pharmacy Address 2

Pharmacy City

Pharmacy State

Pharmacy Zip Code

Drug Enforcement Agency (DEA) Registration Number

Prescriber NPI

Prescriber Last Name

Prescriber First Name & M.I.

Prescriber Address 1

Prescriber Address 2

Prescriber City

Prescriber State

Prescriber Zip Code

Prescriber Specialty

Transaction Number

Message

Plan Name/Group Name

Effective Date

Contact/Information Source

Maximum Number of Transactions Supported per transmission

Reversal Window

COB Processing

BIN Number

Version/Release Number

Transaction Code

Processor Control Number

Transaction Count

Service Provider ID Qualifier

Service Provider ID

Date of Service

Software Vendor/Certification ID

Segment Identification

Patient ID Qualifier

Patient ID

Date of Birth

Patient Gender Code

Place of Service

Patient First Name

Patient Last Name

Patient Street Address

Patient City Address

Patient State/Province Address

Patient Zip/Postal Zone

Patient Phone No.

Employer ID

Pregnancy Indicator

Patient Email Address

Patient Residence

Segment Identification

Provider ID Qualifier

Provider ID

Segment Identification

Prescriber Qualifier

Prescriber ID

Prescriber Last Name

Prescriber Phone Number

Primary Care Provider ID Qualifier

Primary Care Provider ID

Primary Care Provider Last Name

Prescriber First Name

Prescriber Street Address

Prescriber City Address

Prescriber State/Providence Address

Prescriber Zip/Postal Zone

Cardholder ID

Cardholder First Name

Cardholder Last Name

Home Plan

Plan ID

Eligibility Clarification Code

Facility ID

Group ID

Person Code

Patient Relationship Code

Medicaid Indicator

Provider Accept Assignment Indicator

CMS Part D Defined Qualified Facility

Medicaid ID Number

Medicare Agency Number

Prescription/Service Ref No. Qualifier

Prescription/Service Ref No.

Product/Service ID Qualifier

Product/Service ID

Associated Prescription/Service Ref No.

Associated Prescription/Serv. Date

Procedure Modifier Code Count

Procedure Modifier Code

Quantity Dispensed

Fill Number

Days Supply

Compound Code

DAW/Prod Selection Code

Date Prescription Written

Number of Refills Authorized

Prescription Origin Code

Submission Clarification Code Count

Submission Clarification Code

Other Coverage Code

Special Packaging Indicator

Orig Prescribed Prod/Sery ID Qualifier

Orig Prescribed Prod/Sery Code

Originally Prescribed Quantity

Unit of Measure

Level of Service

Prior Authorization Type Code

Prior Authorization No. Submitted

Intermediary Authorization Type ID

Intermediary Authorization ID

Dispensing Status

Quantity Intended to be Dispensed

Days Supply Intended to be Dispensed

Delay Reason Code

Patient Assignment Indicator

Route of Administration

Compound Type

Pharmacy Service Type

Date of Injury

Employer Name

Employer Street Address

Employer City Address

Employer State/Province Address

Employer Zip/Postal Zone

Employer Phone Number

Employer Contact Name

Carrier ID

Claim Reference/ID

Billing Entity Type Indicator

Pay To Qualifier

Pay To ID

Pay To Name

Pay To Street Address

Pay To City

Pay To State/Province Address

Pay To Zip/Postal Zone

Generic Equivalent Product ID Qualifier

Generic Equivalent Product ID

Coordination of Benefits/Other Payments Count

Other Payer Coverage Type

Other Payer ID Qualifier

Other Payer ID

Other Payer Date

Other Payer Amount Paid Count

Other Payer Amount Paid Qualifier

Other Payer Amount Paid

Other Payer Reject Count

Other Payer Reject Code

Internal Control Number

Other Payer—Patient Responsibility Amount Count

Other Payer—Patient Responsibility Amount Qualifier

Other Payer—Patient Responsibility Amount

Benefit Stage Count

Benefit Stage Qualifier

Benefit Stage Amount

DUR/PPS Code Counter

Reason for Service Code

Professional Service Code

Result of Service Code

DUR/PPS Level of Effort

DUR Co-Agent ID Qualifier

DUR Co-Agent ID

Compound Dosage Form Description Code

Compound Dispensing Unit Form Indicator

Compound Ingredient Component Count

Compound Product ID Qualifier

Compound Product ID

Compound Ingredient Quantity

Compound Ingredient Drug Cost

Compound Ingredient Basis of Cost Determination

Compound Ingredient Modifier Count

Compound Ingredient Modifier

Coupon Type

Coupon Number

Coupon Value Amount

Ingredient Cost Submitted

Dispensing Fee Submitted

Incentive Amount Submitted

Other Amount Claimed Submitted Count

Other Amount Claimed Submitted Qualifier

Other Amount Claimed Submitted

Flat Sales Tax Amount Submitted

Percentage Sales Tax Amount Submitted

Percentage Sales Tax Rate Submitted

Percentage Sales Tax Basis Submitted

Usual and Customary Charge

Gross Amount Due

Basis of Cost Determination

Diagnosis Code Count

Diagnosis Code Qualifier

Diagnosis Code

Clinical Information Counter

Measurement Date

Measurement Time

Measurement Dimension

Measurement Unit

Measurement Value

Table 2

The BIN associated with the system 600 causes the transmission from the pharmacy system 610 to be routed to the system 600. In an embodiment, the transmission is routed directly to the system 600. In another embodiment, the transmission from the pharmacy system 610 is routed to a switch. A switch is an entity that routes claims from the pharmacy system 610 to the destination, where the destination is indicated by the BIN. The switch receives the transmission from the pharmacy system 610 and routes the transmission to the destination specified by the BIN.

The system 600 receives the data packet from the pharmacy system 610 and processes the drug pricing request. In one embodiment, the system 600 uses some or all elements of the data packet to create a new data packet to send to the PBM systems 290A, 290B and insurance pricing system 440. In one embodiment, the system 600 may create a different data packet for each pricing request sent to a different PBM system 290A, 290B and/or insurance pricing system 440.

The system 600 obtains drug pricing information from multiple PBM systems 290A, 290B, as described above and illustrated in FIGS. 2 and 4. The system 600 may obtain the drug pricing information through the APIs 630d, 630e. The multiple PBM systems 290A, 290B may each provide an API, which includes functions that can be called by the system 600. The system 600 can call various functions to obtain relevant information. The information processed by the system 600 from the PBM systems 290A, 290B can be stored as pricing information 621 in the database 620.

The system 600 may obtain drug pricing for a plurality of drugs from the multiple PBMs 290A, 290B before the transmission from the pharmacy system 610 is received and store the pricing information 621 in the database 620. The system 600 may update or revise the pricing information 621 from the multiple PBMs 290A, 290B at scheduled intervals as described above. In an embodiment, the system 600 may obtain drug pricing for the requested drug in the transmission from the multiple PBMs 290A, 290B after receiving the transmission from the pharmacy system 610.

The system 600 obtains insurance drug pricing information from the insurance pricing system 440, as described above and illustrated in FIG. 4. The system 600 may obtain the drug pricing information through the API 630f. The insurance pricing system 440 may provide an API, which includes functions that can be called by the system 600. The system 600 can call various functions to obtain relevant information. The information processed by the system 600 from the insurance pricing system 440 can be stored as pricing information 621 in the database 620.

In an embodiment, the system 600 retrieves the user profile 622 based on the member ID in the transmission and retrieves the user's insurance information from the user profile 622. The user may not have submitted insurance information when registering the user profile, or may not have updated the user profile with insurance information. When there is no insurance information retrieved from the user profile 622, the system 600 processes the drug pricing information from the multiple PBMs 290A, 290B and returns the lower PBM rate to the pharmacy system 610.

When the user profile 622 includes insurance information, the system 600 verifies the user's insurance. In an embodiment, the system 600 sends a message to the insurance pricing system 440 requesting verification of the user's insurance and/or coverage of the requested drug. The system 600 further requests the user's insurance price for the drug from the insurance pricing system 440. The system 600 may request information relating to the deductible associated with the user's insurance. The system 600 may request information relating to whether additional approvals and/or steps are necessary to obtain the drug and/or drug pricing, such as whether a drug requires prior authorization.

The insurance pricing system 440 may respond with one or more of a confirmation that the user/requested drug is covered by the insurance pricing system 440, the user's insurance price for the drug, the user's deductible, whether the drug requires prior authorization, the amount of the deductible that is satisfied, and the like.

The system 600 may obtain insurance drug pricing for a plurality of drugs from the insurance pricing system 440 before the transmission from the pharmacy system 610 is received and store the pricing information 621 in the database 620. The system 600 may update or revise the insurance pricing information 621 from the insurance pricing system 440 at scheduled intervals. In an embodiment, the system 600 may obtain insurance drug pricing for the requested drug in the transmission from the insurance pricing system 440 after receiving the transmission from the pharmacy system 610.

In an embodiment, the system 600 retrieves the user profile 622 based on the member ID in the transmission. In an embodiment, the system 600 determines based on information from the user profile 622 if a user is eligible for a patient assistance discount.

The system 600 processes the drug pricing information 621 received from the multiple PBMs 290A, 290B and the insurance pricing system 440. For example, the system 600 compares the insurance price and the lower PBM rate from the multiple PBMs 290A, 290B. The system 600 retrieves the pricing profile from the user profile 622.

In one embodiment, when no pricing profile is stored in the user profile 622, the system 600 returns the insurance price to pharmacy system 610 if insurance pricing is available. If insurance pricing is not available, in one embodiment, the system 600 returns the lower PBM price to the pharmacy system 610. If insurance pricing is not available, in one embodiment, the system 600 returns an unfunded price to the pharmacy system 610.

In one embodiment, if a user is eligible for a patient assistance discount, the system 600 returns information to the pharmacy system 610 allowing the patient assistance discount to be applied at the pharmacy system 610. In one embodiment, if the patient assistance discount requires that a user have commercial insurance, the system 600 may provide to the pharmacy system 610 the insurance information such as insurance company, group number, member ID associated with the insurance company and the patient assistance discount information such as a BIN, PCN, group number and member ID associated with the patient assistance discount.

When the pricing profile indicates the user prefers to use the lowest price, the system 600 returns the lower of the insurance pricing and the lower PBM pricing to the pharmacy system 610.

When the pricing profile indicates the user prefers to use the insurance to fill prescriptions and the insurance price is lower than the lower price from the multiple PBMs 290A, 290B, the system 600 returns the insurance price to the pharmacy system 610.

When the pricing profile is to use the insurance to fill prescriptions and the insurance price is higher than the lower price from the multiple PBMs 290A, 290B, the system 600 sends a digital alert 602 to the user via the user interface 640. The digital alert 602 notifies the user of the lower PBM price for the drug.

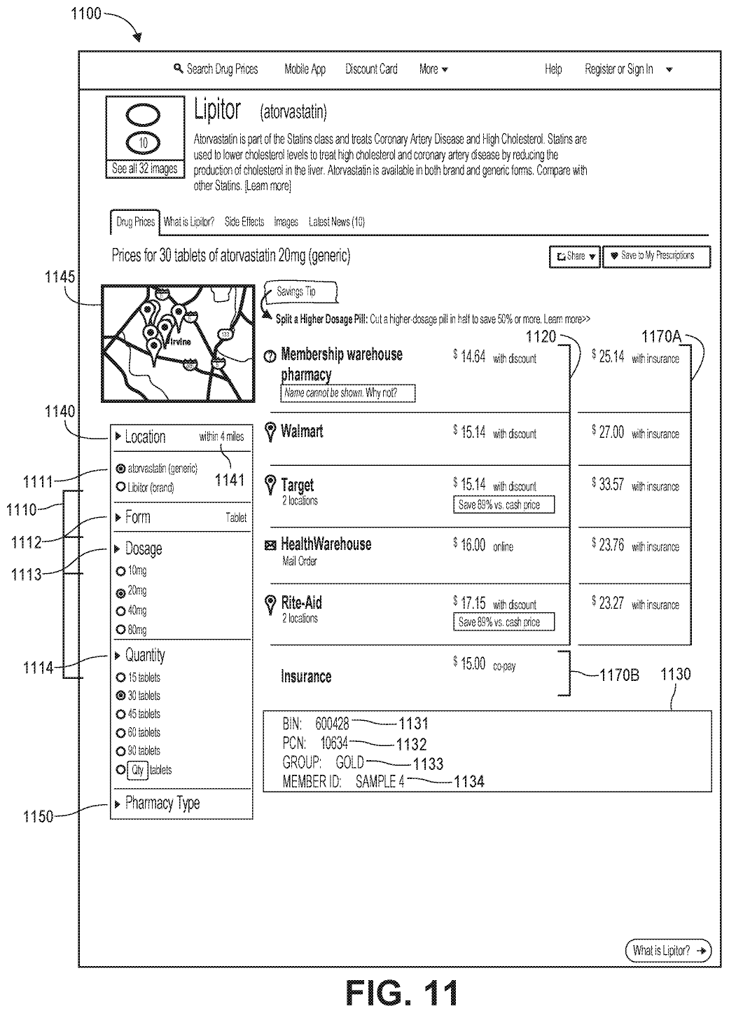

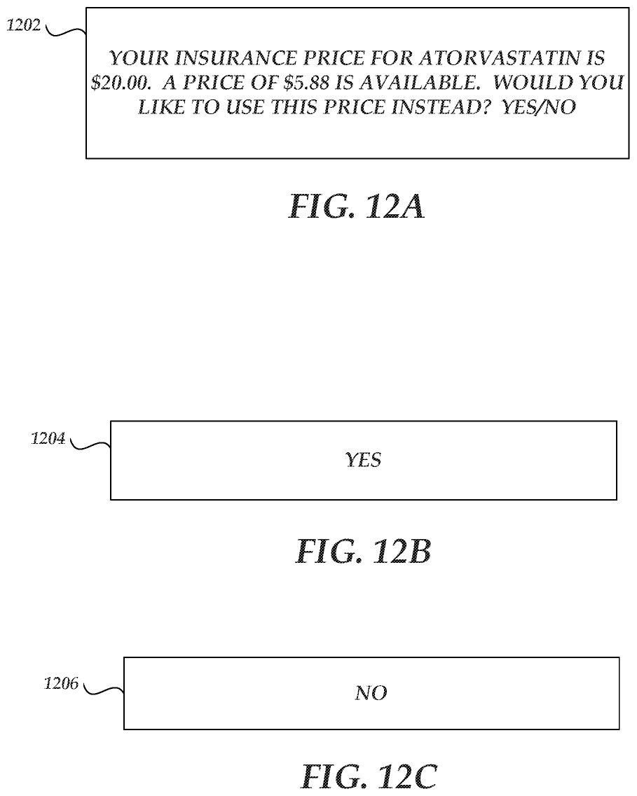

The digital alert 602 may be a text message, a push notification, a SMS message, an email, etc., that is sent to the user's mobile phone 111, smartphone, computer 112, tablet 113, laptop 114, etc. In an embodiment, the user's preferred communication is stored in the user profile 622. An example of a text message digital alert 602 is illustrated in FIG. 12A. Message to the user may also be sent via direct mail.

In other embodiments, the digital alert or the message may be a reminder or recommendation from the system 600 to the user. Examples of message types and message content are, but not limited to, (1) reminder to pick up a prescription; (2) recommendation to transfer the prescription to another pharmacy; (3) recommendation for a generic alternative; (4) recommendation for an alternative therapeutic; (5) recommendation for a different day supply; (6) recommendation for a different dosage, different form, and/or different quantity, etc.; (7) information concerning the user's insurance deductible; (8) an amount the user has spent toward the deductible; (9) the difference between what has been spent and the user's maximum out of pocket expenses; (10) refill reminder; (11) reminder to obtain a new prescription; (12) recommendations to use a patient assistance discount and the like.

The system 600 reformats the pricing information 621 from the database 620 into the format appropriate for transmission of the digital alert 602. For example, the system 600 reformats the pricing information 621 to a format that can be sent in a text message to the user's smartphone.

The system 600 receives a response 604 from the user via the user interface 640. The response 604 may be a text message, a response to a push notification a SMS message, an email, etc., that is sent from the user's mobile phone 111, smartphone, computer 112, tablet 113, laptop 114, etc. The response 604 may indicate that the user prefers to use the insurance to fill the prescription. In that case, the system 600 returns the insurance price for the drug to the pharmacy system 610.

The response 604 may indicate that the user prefers the lower price. When the response 604 indicates that the user prefers the lower price, the system 600 updates the user profile to indicate that the user prefers the lower price, and returns the lower PBM price for the drug to the pharmacy system 610. Examples of text message responses 604 are illustrated in FIGS. 12B and 12C.

The system 600 reformats the response 604 from the format of the response transmission into the format appropriate update the user profile 622 to be stored in the database 620. For example, the user interface 640 returns a text message from the user's smartphone. The system 600 reformats pricing profile indicated in the text message to a format that can be stored in the database 620 in the user profile 622.

In an embodiment, the system 600 may return the insurance price for the drug to the pharmacy system 610 before sending the digital alert to the user interface 640. If the system 600 receives the response within a predetermined time and the response indicates a change in the user pricing preference from the insurance price to the lowest price, the system 600 may send a second message for the current prescription fill to the pharmacy system 610 that returns the lower PBM price. The system 600 updates the user profile 622 with the user pricing preference from the response.

If the response is not received within the predetermined time and the response indicates a change in the user pricing preference from the insurance price to the lowest price, the system 600 updates the user profile 622 with the user pricing preference from the response and uses the updated user pricing preference for future fills of the prescription.

As described above, the system 600 returns the drug price to the pharmacy system 610. In an embodiment, the syntax of a transmission request and response is illustrated in TABLE 1.

In an embodiment, the transmission from the system 600 to the pharmacy system 610 may comprise one or more of the following data fields:

Copay Amount

Submitted Amount

Patient Cost Savings

Patient Percentage Savings

Table 3

The pharmacy system 610 receives the transmission from the system 600 and charges the returned drug price to the user.

In some embodiments, the system 600 is integrated into an electronic medical record (EMR) system. The system 600 can provide an API that can be called by the EMR system. For example, the EMR system calls one or more functions of the API to determine refill status of the user's prescriptions, which may provide an indication of patient compliance.

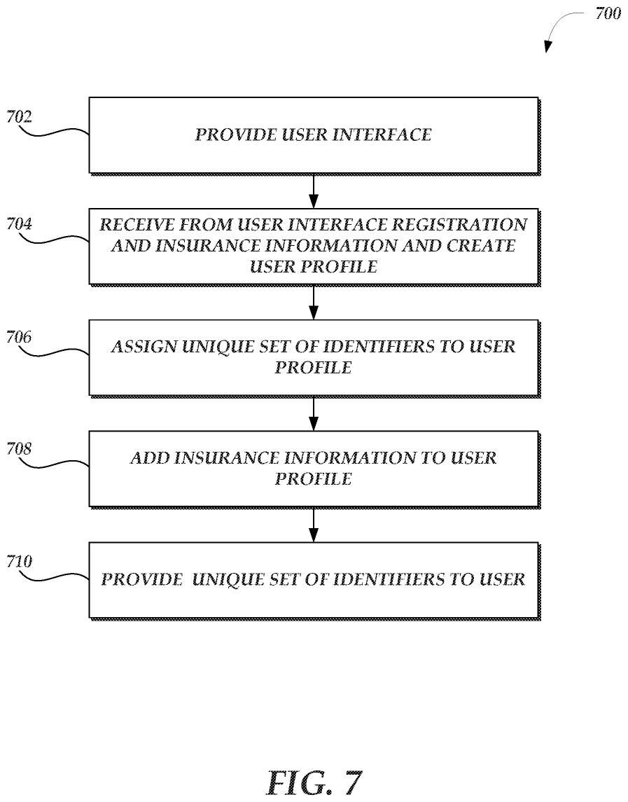

FIG. 7 illustrates an example process 700 to generate the user profile 622. The process 700 is described with respect to the system 600 of FIG. 6. However, one or more of the steps of process 700 may be implemented by other systems, such as the system 100 illustrated in FIG. 1, the system 200 illustrated in FIG. 2, and the system 400 illustrated in FIG. 4. The process 700 can be implemented by any one of, or a combination of, the components of the system 600.

In an embodiment, the user profile 622 is created via the user interface 640 in the system 600. At block 702 the system 600 provides the user interface 640. At block 704, the system 600 receives information input in the user interface 640. The information may comprise of user information, such as user name, mailing address, mobile number, email address, insurance information such as insurance company, group number, member ID associated with the insurance company, and user's prescriptions. The information may also include whether the user prefers to fill prescriptions using the insurance or using the lowest price, preferred method of contact, preferred form of drug, etc. The system 600 creates a user profile 622 based at least in part on the received information. In an embodiment, the system 600 may generate a user profile without user input.

At block 706, the system 600 assigns a unique set of identifiers to the user profile 622 in response to the provided user and insurance information. The user profile may include the user name and the unique set of identifiers (including BIN, PCN, group number and member ID associated with the system 600). At block 708, the system 600 stores the insurance information in a user profile 622 and stores the user profile 622 in the database 620. At block 710, the system 600 provides the user the unique set of identifiers. In an embodiment, the system 600 may present the user with the unique set of identifiers via the user interface 640. The unique set of identifiers may be presented as a digital discount card, via website, mobile application, text or email. The discount card may also be mailed to the user. An example of the unique set of identifiers on a discount card is illustrated in FIG. 9.

The process 700 can include fewer, more, or different blocks than those illustrated in FIG. 7 without departing from the spirit and scope of the description. Moreover, it will be appreciated by those skilled in the art and others that some or all of the functions described in this disclosure may be embodied in software executed by one or more processors of the disclosed components and mobile communication devices. The software may be persistently stored in any type of non-volatile storage.

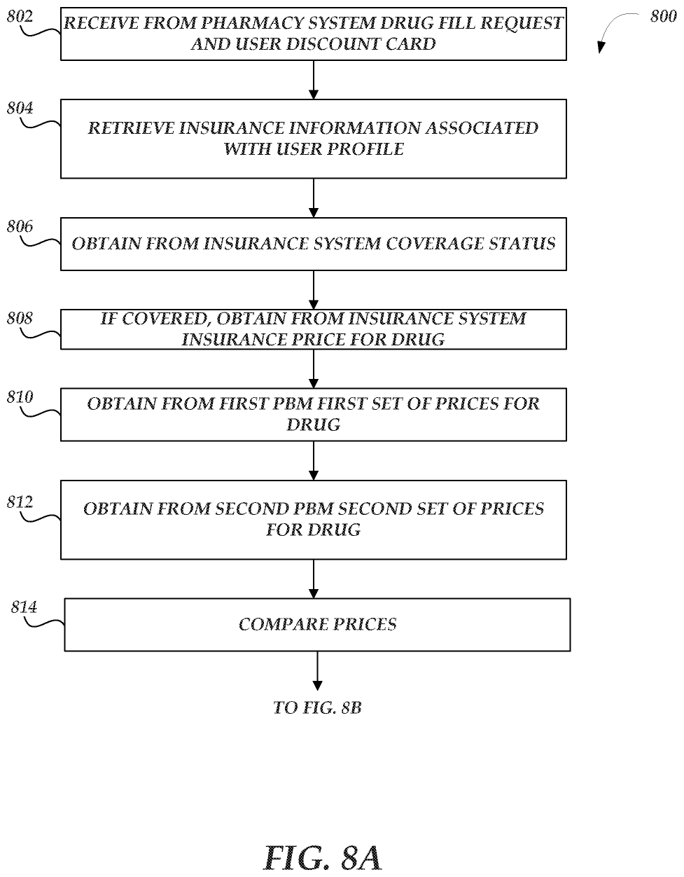

FIGS. 8A and 8B illustrate an example process 800 to provide drug prices in response to a user profile. The process 800 is described with respect to the system 600 of FIG. 6. However, one or more of the steps of process 800 may be implemented by other systems, such as the system 100 illustrated in FIG. 1, the system 200 illustrated in FIG. 2, and the system 400 illustrated in FIG. 4. The process 800 can be implemented by any one of, or a combination of, the components of the system 600 (e.g., the PBM module 650, the insurance module 660, and the pharmacy module 670). Further details regarding certain aspects of at least some of steps of the process 800 are described in greater detail above with reference to FIGS. 1-7.

The system 600 can communicate with the pharmacy system 610 through the API 630a. At block 802, the system 600 receives the data packet from the pharmacy system 610. The data packet may comprise the data fields in TABLE 2 according to the syntax of TABLE 1 and includes at least the unique BIN associated with the system 600 and a drug pricing request to fill a prescription. The transmission including the data packet from the pharmacy system 610 is routed according the BIN that the pharmacy system 610 retrieved from the user's unique set of identifiers, presented in the form of a discount card. The BIN is associated with the system 600 and causes the switch to route the drug pricing request to the system 600.

The system 600 can communicate with the insurance pricing system 440 through the API 630f. At block 804, the system 600 retrieves the insurance information from the transmission from the pharmacy system 610. At block 806, the system 600 obtains the status of the insurance coverage from the insurance pricing system 440. In an embodiment, the status comprises at least one of whether the user is covered under the insurance pricing system 440 and whether the requested drug is covered under the insurance pricing system 440. When coverage exists, the system 600, at block 808, obtains the insurance price for the drug from the insurance pricing system 440, as described above and illustrated in FIGS. 4-6. The system 600 stores the pricing information 621 from the insurance pricing system 440 in the database 620.

The system 600 can communicate with the first PBM 290A through the API 630d. At block 810, the system 600 obtains a first set of prices for the drug from the first PBM 290A, as described above and illustrated in FIGS. 1-6. The system 600 can communicate with the second PBM 290B through the API 630e. At block 812, the system 600 obtains a second set of prices for the drug from the second PBM 290B, as described above and illustrated in FIGS. 1-6. The system 600 stores the pricing information 621 from the multiple PBMs 290A, 290B in the database 620.

At block 814, the system 600 processes the pricing information 621. For example, the system 600 compares the pricing information 621 from the first PBM 290A with the pricing information 621 from the second PBM 290B and also compares the pricing information 621 from the multiple PBMs 290A, 290B with the pricing information 621 from the insurance pricing system 440. Based on the comparisons, the system determines a lower price for the requested drug.

At block 816, the system 600 retrieves the user profile 622 stored in the database 620. In an embodiment, the system 600 created the user profile 622 from the registration information and the insurance information provided by the user. If, at block 818, there are no user preferences as to whether the user prefers to fill the prescription using the insurance, in one embodiment, the system 600 returns insurance pricing if available. If, at block 818, there are no user preferences as to whether the user prefers to fill the prescription using the insurance, in another embodiment, the system 600 returns unfunded pricing if available. If not available, the system 600 returns the lower PBM price.

At block 820, when the user profile 622 indicates that the user prefers to use the lowest price, the system 600 returns the lower of the insurance price and the lower PBM price when the insurance price is available. If not available, the system 600 returns the lower PBM price to the pharmacy system 610.

At block 822, when the user profile 622 indicates that the user prefers to use the insurance to fill the prescription, based on the comparisons from block 814, determines whether the insurance price for the drug is higher or lower than the lower PBM price. When the insurance price is lower than the lower PBM price, the system 600 returns the insurance price to the pharmacy system 610.

The system 600 can communicate with the user interface 640 through the APIs 630a and 630b. At block 824, when the insurance price is more than the lower PBM price, the system 600 sends a digital alert 602 to the user interface 640. In an embodiment, the insurance price is more than the lower PBM price when the insurance price is greater than a predetermined amount, greater than a percentage, etc., than the lower PBM price. The digital alert may be an electronic message, such as a text message, push notification, SMS message, email, etc. sent to the user interface 640 on an electronic device, such as a mobile phone 111, smartphone, computer 112, tablet 113, laptop 114, etc. The system 600 reformats the pricing information 621 to a format that is appropriate for transmission in the digital alert 602. The digital alert 602 notifies the user that the insurance price for the drug is more than an available PBM unfunded price for the drug.

At block 826, the system 600 receives the response 604 from the user interface 640. The response 604 notifies the system 600 of the user's pricing profile. For example, the user may prefer to pay the lower price or may prefer to fill the prescription using the insurance. The response 604 may be an electronic message, such as a text message, push notification, SMS message, email, etc. sent from the user interface 640 on an electronic device, such as a mobile phone 111, smartphone, computer 112, tablet 113, laptop 114, etc.

The system 600 reformats the user pricing profile 621 to a format that is appropriate the user profile 622 stored in the database 620. At block 828, the system 600 updates the user profile with any changes to the user's pricing preference.

At block 830, the system 600 returns the drug price to the pharmacy system 610. In an embodiment, the system 600 returns the drug price to the pharmacy system 610 according the updated user profile 622. In another embodiment, the system 600 returns to the pharmacy system 610 one or more of a drug price and a message. For example, if the response indicates that the user prefers to use the insurance, the system 600 returns the insurance price to the pharmacy system 610. If the response indicates that the user prefers to use the lowest price, the system 600 returns the lower PBM price to the pharmacy system 610.

As described above in reference to FIG. 6, the system 600, at block 822, may return the insurance price for the drug to the pharmacy system 610 before sending the digital alert to the user interface 640. If the system 600, at block 826, receives the response within a predetermined time and the response indicates a change in the user pricing preference from the insurance price to the lowest price, the system 600 may send a second message for the current prescription fill to the pharmacy system 610 that returns the lower PBM price. The system 600 updates the user profile 622 with the user pricing preference from the response.

If, at block 826, the response is not received within the predetermined time and the response indicates a change in the user pricing preference from the insurance price to the lowest price, the system 600 updates the user profile 622 with the user pricing preference from the response and uses the updated user pricing preference for future refills of the prescription.

The process 800 can include fewer, more, or different blocks than those illustrated in FIGS. 8A and 8B without departing from the spirit and scope of the description. Moreover, it will be appreciated by those skilled in the art and others that some or all of the functions described in this disclosure may be embodied in software executed by one or more processors of the disclosed components and mobile communication devices. The software may be persistently stored in any type of non-volatile storage.

FIG. 9 illustrates an example discount card 900 with the unique set of identifiers assigned to a user profile. In the illustrated example, the membership card represents a user profile in the system 600 and includes the BIN associated with the system 600, the PNC associated with the system 600, the group number associated with the system 600 and the member ID associated with the system 600. In other embodiments, the discount card 900 may include more or less information. The discount card 900 may be presented in digital form via the user interface 640, via the pharmacy system 610, or in physical form.

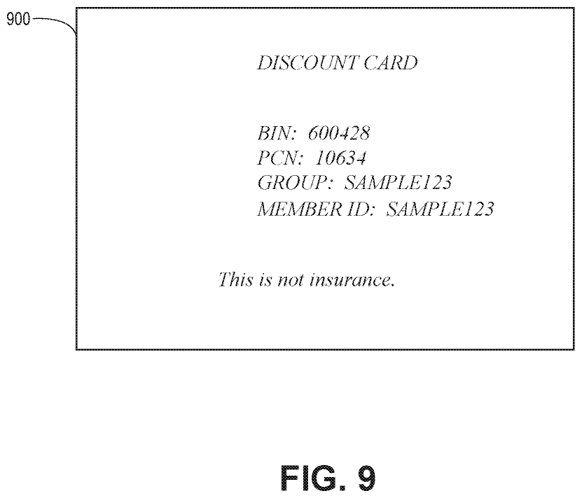

FIG. 10 illustrates an example of a user interface 1000 to provide drug prices from various PBMs and the user's insurance price provider. Details relating to the user interface 1000 are explained in more detail with respect to FIGS. 1-9. Various functions relating to the user interface 1000 can be performed by the system 100 illustrated in FIG. 1, the system 200 illustrated in FIG. 2, the system 400 illustrated in FIG. 4, the system 600 illustrated in FIG. 6, or any other appropriate system.

The user interface 1000 is provided to receive information relating to a drug from or associated with a user. The user interface 1000 may be a web user interface. The user interface 1000 can include a section for entering the drug information 1010. In one embodiment, the section 1010 includes an input field for drug name 1011. In another embodiment the section 1010 further includes an input field for location 1012. The section 1010 also includes a button 1013 for generating a request for pricing information for the drug name 1011 entered by the user. The drug name field 1011 can display the name of a common drug as a suggestion to provide users with some guidance on entering drug names. In one embodiment, the user can enter a health condition (e.g., asthma) in the drug name field 1011 to view prices for drugs related to the health condition. In some embodiments, as the user types the drug name, the drug name field 1011 shows a list of drug names that include the typed letters and/or shows related drug names or conditions for the entered drug name. The location input field 1012 can accept various types of input, such as city, zip code, address, etc.

In an embodiment, the user interface 1000 is provided to receive information relating to the user's insurance. The user interface 1000 can include a section for entering the insurance information 1020. In one embodiment, the section 1020 includes an input field for an insurance carrier 1021, a plan number 1022, a member ID from the insurance carrier for the insured 1023, and the insured's name 1024.

The example user interface 1000 in FIG. 10 may also include various menu items. Some examples include a menu item for downloading a mobile application for requesting drug prices from the system 200, 400, 600, a menu item for applying for a discount card, a menu item for registering to become of a member of the system 200, 400, 600, and a menu item for signing in for registered members. The system 200, 400, 600 can also provide additional features, such as providing price alerts, prescription fill alerts, etc.

The system 200, 400, 600 can store information for registered members, including the user information, contact information, unique set of identifiers, drug pricing preference and prescription information, such as drugs purchased by members provided by the system 200, 400, 600. The information relating to drugs purchased by members may be stored in the user profile. In an embodiment, the information relating to drugs purchased may include refill information. The system 200, 400, 600 may store one or more of the date, quantity, strength, etc. of a refill drug purchased by the user. For example, a physician may prescribe a drug for a chronic illness and user may not refill the prescription. The system 200, 400, 600 may store the refill information and use the refill information to provide an indication of user compliance with the drug usage as specified by the physician on the prescription.

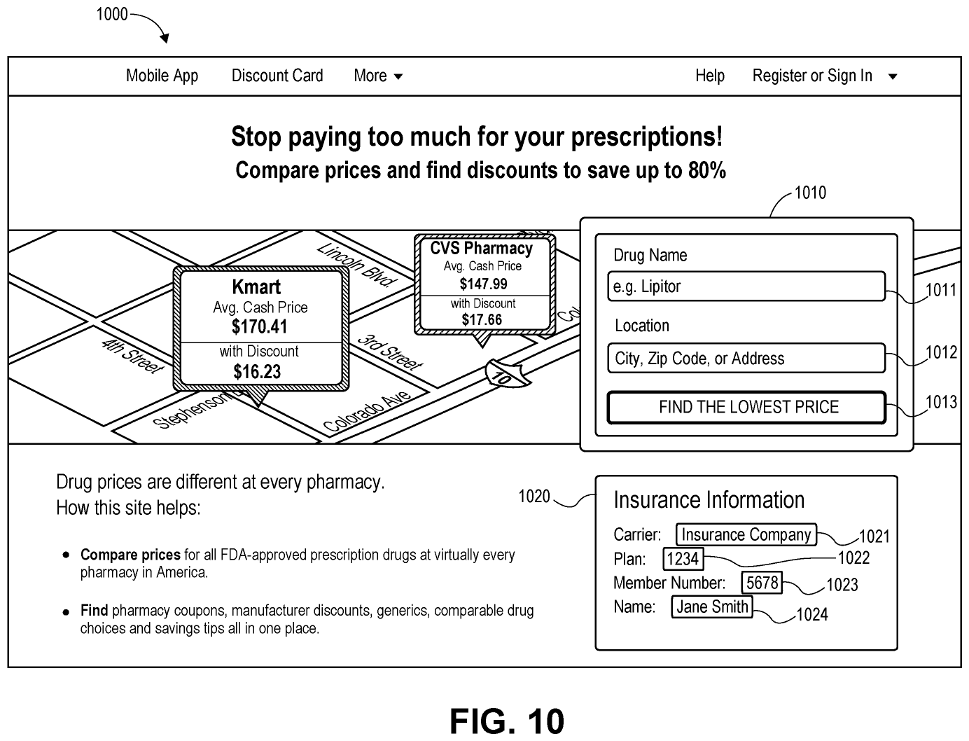

FIG. 11 illustrates an example of a user interface 1100 to provide drug prices from the insurance price provider and various PBMs. Details relating to the user interface 1100 are explained in more detail with respect to FIGS. 1-10. Various functions relating to the user interface 1100 can be performed by the system 100 described in FIG. 1, the system 200 described in FIG. 2, the system 400 described in FIG. 4, the system 600 described in FIG. 6, or any other appropriate system. The user interface 1100 is provided to display drug prices and/or information relating to a specific drug. As explained with respect to FIG. 10, the user may enter the drug name, the insurance information, and the location information and send a request for pricing information for a specific drug. In response to the request, the user interface 800 can provide drug prices for the drug. The user interface 1100 may display drug information 1110, such as the name of the drug 1111, the form of the drug 1112, the dosage of the drug 1113, the quantity of the drug 1114, etc. The name of the drug 1111 can be the generic name, brand name, or both. The drug information 1110 can be for a specific package of the drug (e.g., the most common package).

When there is more than one type of package available for a drug, the user interface 1100 can display various options for indicating a particular package of the drug. For example, the system 200, 400, 600 can provide options for generic or brand version 1111, options for form 1112, options for dosage 1113, options for quantity 1114, etc. The user can select an appropriate option for each category to select the package of the drug that the user wants. In the example of FIG. 11, atorvastatin is available as generic or brand version, and the generic name and the brand name of the drug are provided as options for the drug name 1111. The only option for form 1112 is tablet. The options for dosage 1113 are 10 mg, 20 mg, 40 mg, and 80 mg. The options for quantity 1114 are 15 tablets, 30 tablets, 45 tablets, 60 tablets, and 90 tablets. The user interface 1100 also provides an input field for entering a desired quantity 1114. The options corresponding to the package for which the prices are currently displayed are selected under each category. In this example, the prices 1120 are for the generic version atorvastatin in tablet form having 20 mg dosage in quantity of 30 tablets, and these options are marked in the user interface 1100. The user can select any of the options provided under each category to designate a combination of options that the user is interested in.

The user interface 1100 may also display prices 1120 for the drug available from one or more pharmacies. The prices 1120 may be for the most common or user selected package for the drug. In one embodiment, the user interface 1100 displays a default number of prices, such as portion of the drug pricing information returned from the PBMs. In the example of FIG. 11, each price 1120 is associated with one pharmacy. If there is more than one price for each pharmacy, the multiple prices for the pharmacy may have been ranked according to a lowest to highest algorithm. The system 200, 400, 600 can display one price for each pharmacy, for example, the highest ranked price. The price 1120 for Walmart is $15.14 with discount. The price 1120 for Target is also $15.14 with discount. The price 1120 for Health Warehouse, an online pharmacy, is $16.00 without a discount. In the example of FIG. 11, the prices 1120 from different pharmacies are listed in the order of lowest to highest.

The user interface 1100 may also display the unique set of identifiers for a member 1130 if the user is a member of the system 200, 400, 600. The membership information may include the BIN 1131 and PCN 1132 associated with the system 200, 400, 600. The membership information may further include the group number 1133 and the member ID 1134 associated with the user.

The user interface 1100 may also display prices 1170A, 1170B for the drug through the user's insurance company. Typically, the user interface 1100 displays prices 1170A or price 1170B. Prices 1170A indicate the insurance price of the drug as negotiated by each of the various pharmacies. The negotiated insurance price 1170A for Walmart is $25.14. The negotiated insurance price 1170A for Target is $27.00. The negotiated insurance price 1170A for Health Warehouse is $23.76.

Price 1170B indicates the amount of the user's co-pay when the user's insurance covers the cost of prescriptions less the co-pay amount. The price 1170A, 1170B may be for the most common or user selected package for the drug.

The user interface 1100 can also display the location information 1140 relating to the prices 1120. The location information 1140 can be entered by the user. The user interface 1100 may also indicate the default radius 1141 for including pharmacy locations relating to the location information 1140. In FIG. 11, the default radius 1141 is listed as 4 miles. The user can change the radius 1141 as appropriate. For example, the user can select from a list of options in a drop-down menu. The user may be able to change the location information 1140 after the prices 1120 are initially displayed, and the displayed prices 1120 can change based on the location information 1140. The user interface 1100 can provide an input field for entering a new city, zip code, address, etc. The user interface 1100 may also provide a map 1145 showing a region that includes the pharmacy locations for the prices 1120 displayed in the user interface 1100. As the user updates the location information 1140 or the default radius 1141, the map 1145 can change to reflect the updated information.

The user interface 800 may also display pharmacy options 1150. As explained above, the user can choose which types of pharmacies to view prices for. Some examples of pharmacy options or types include: 24-hour, mail order, home delivery, compounding, walk-in clinic, drive-up window, languages spoken, major chains, etc.

The example user interface 1100 in FIG. 11 may also include other types of information relating to the drug, such as a description of the drug, side effects, images of the drug, news relating to the drug, advertisements relating to the drug, etc. The user interface 1100 can also provide other useful information, such as savings tips.

FIG. 12A illustrates an example of a digital alert 1202 provided to the user interface 640. The system 600 sends the digital alert 1202 to the user when an insurance price is returned at the pharmacy and the system 600 obtains a lower drug price from at least one PBM. The digital alert 1202 may be a text message, push notification, a SMS message, an email, or an alert via the user interface 640. The system 600 sends the digital alert 1202 to a user device. For example, the user may provide a cell phone number and the digital alert 1202 is texted to the user's cell phone or smartphone. In another embodiment, the user provides the system 600 with an email address, and the digital alert 1202 is emailed to the user. Other examples of user devices where the user can receive the digital alert 1202 and send a response to the digital alert are, but not limited to, mobile phone 111, computer 112, tablet 113, laptop 114, and the like.

The example digital alert 1202 illustrated in FIG. 12 is a text message sent to the user's smartphone and states “YOUR INSURANCE PRICE FOR ATORVASTATIN IS $20.00. A PRICE OF $5.88 IS AVAILABLE. WOULD YOU LIKE TO USE THIS PRICE INSTEAD? YES/NO.” Another example of a text message sent to the user's smartphone is “YOU REQUESTED THAT WE FILL YOUR PRESCRIPTION FOR ATORVASTATIN USING THE INSURANCE INFORMATION THAT YOU PROVIDED. A LOWER PRICE IS AVAILABLE. DO YOU WANT TO USE THE LOWER PRICE? YES/NO”

FIGS. 12B and 12C illustrate examples of user responses 1204, 1206 sent from the user interface 640 in response to receiving the digital alert 1202. For example, the user may want to fill the prescription with the insurance coverage and replies with response 1204, “NO” sent from the user device. In another example, the user may want the lowest price for the drug and replies with the response 1206, “YES”, sent from the user device. The system 600 receives the response and updates the pricing profile in the user profile.

### Terminology

The various illustrative processes described herein may be implemented as electronic hardware, computer software, or combinations of both. To clearly illustrate this interchangeability of hardware and software, various illustrative components, blocks, modules, and states have been described above generally in terms of their functionality. However, while the various modules are illustrated separately, they may share some or all of the same underlying logic or code. Certain of the logical blocks, modules, and processes described herein may instead be implemented monolithically.

The various processes described herein may be implemented or performed by a machine, such as a computer, a processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor, a controller, microcontroller, state machine, combinations of the same, or the like. A processor may also be implemented as a combination of computing devices, e.g., a combination of a DSP and a microprocessor, a plurality of microprocessors or processor cores, one or more graphics or stream processors, one or more microprocessors in conjunction with a DSP, or any other such configuration.

The processes described herein may be embodied directly in hardware, in a software module executed by a processor, or in a combination of the two. For example, each of the processes described above may also be embodied in, and fully automated by, software modules executed by one or more machines such as computers or computer processors. A module may reside in a computer-readable storage medium such as RAM memory, flash memory, ROM memory, EPROM memory, EEPROM memory, registers, hard disk, a removable disk, a CD-ROM, memory capable of storing firmware, or any other form of computer-readable storage medium known in the art. An exemplary computer-readable storage medium can be coupled to a processor such that the processor can read information from, and write information to, the computer-readable storage medium. In the alternative, the computer-readable storage medium may be integral to the processor. The processor and the computer-readable storage medium may reside in an ASIC.

Depending on the embodiment, certain acts, events, or functions of any of the processes or algorithms described herein can be performed in a different sequence, may be added, merged, or left out. Thus, in certain embodiments, not all described acts or events are necessary for the practice of the processes. Moreover, in certain embodiments, acts or events may be performed concurrently, e.g., through multi-threaded processing, interrupt processing, or via multiple processors or processor cores, rather than sequentially.

Conditional language used herein, such as, among others, “can,” “could,” “might,” “may,” “e.g.,” and from the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

While the above detailed description has shown, described, and pointed out novel features as applied to various embodiments, it will be understood that various omissions, substitutions, and changes in the form and details of the logical blocks, modules, and processes illustrated may be made without departing from the spirit of the disclosure. As will be recognized, certain embodiments of the inventions described herein may be embodied within a form that does not provide all of the features and benefits set forth herein, as some features may be used or practiced separately from others.

1. (canceled)

2. In a drug pricing environment having a drug pricing system, one or more pharmacy benefit managers (PBM), and a pharmacy system, the drug pricing system comprising one or more hardware processors and memory including drug pricing information and user profiles of users associated with the drug pricing system, a method to provide drug pricing to the pharmacy system for a drug requested by a user, the method comprising:
as implemented by one or more hardware processors configured to execute specific instructions stored in the memory;
receiving an electronic transmission comprising at least a routing identifier identifying the drug pricing system as a recipient of the electronic transmission and a drug identifier that identifies a drug;
identifying at least a first price for the drug;
automatically obtaining with the one or more hardware processors at least a second price for the drug from a PBM; and
displaying an electronic notification directly to an individual on an user interface and displaying on such user interface a set of unique identifiers that allow a user to obtain the least one of the first and second prices when the user presents to the set of unique identifiers to a pharmacy system.

3. The method of claim 2, wherein the first price is an insurance price based on insurance information associated with the individual, the insurance price is displayed on the pharmacy system, and the electronic notification with the second price from the PBM is sent directly to a user device for display on the user interface.

4. The method of claim 3, wherein the drug pricing system automatically sends the electronic notification to the user device when the second price from the PBM is less than the insurance price.

5. The method of claim 4, wherein the drug pricing system receives input from the individual that the individual prefers the second price, the drug pricing system sends the second price to the pharmacy system.

6. The method of claim 4, further comprising:
electronically receiving location information associated with a location of the user device;
electronically accessing a geospatial index to identify local pharmacies associated with the location information; and
displaying on a map in the user interface, location information about at least one of the local pharmacies associated with the first and second prices.

7. The method of claim 6 wherein the user interface displays on a map at least the insurance price, and second price with the location information about at least one of the local pharmacies.

8. The method of claim 6 wherein the user interface further displays dosage information.

9. The method of claim 4 further comprising not sending the electronic notification to the individual when past purchases indicate that the user prefers a higher insurance price over a lower second price from the PBM.

10. The method of claim 4 further comprising automatically returning to the pharmacy system the lower of the insurance price and the second price from the PBM when past purchases indicate the user prefers a lowest drug price.

11. The method of claim 3 wherein the electronic notification is at least one of a text message, push notification, and an email, and the user device is a mobile device.

12. A drug pricing system to provide users drug pricing information from multiple drug pricing sources, the drug pricing system comprising:
one or more hardware processors configured to execute specific instructions stored in memory;
the one or more hardware processors receive an electronic transmission comprising at least a routing identifier identifying the drug pricing system as a recipient of the electronic transmission and a drug identifier that identifies a drug;
the one or more hardware processors identify at least a first price for the drug;
the one or more hardware processors automatically obtain at least a second price for the drug from a PBM; and
the one or more hardware processors display an electronic notification directly to an individual on an user interface and displaying on such user interface a set of unique identifiers that allow a user to obtain least one of the first and second prices when the user presents to the set of unique identifiers to a pharmacy system.

13. The drug pricing system of claim 12, wherein the first price is an insurance price based on insurance information associated with the individual, the insurance price is displayed on the pharmacy system, and the electronic notification with the second price from the PBM is sent directly to a user device for display on the user interface.

14. The drug pricing system of claim 13, wherein the drug pricing system automatically sends the electronic notification to the user device when the second price from the PBM is less than the insurance price.

15. The drug pricing system of claim 14, wherein the drug pricing system receives input from the individual that the individual prefers the second price, the drug pricing system sends the second price to the pharmacy system.

16. The drug pricing system of claim 14, further comprising:
electronically receiving location information associated with a location of the user device;
electronically accessing a geospatial index to identify local pharmacies associated with the location information; and
displaying on a map in the user interface, location information about at least one of the local pharmacies associated with the first and second prices.

17. The drug pricing system of claim 16 wherein the user interface displays on a map at least the insurance price, and second price with the location information about at least one of the local pharmacies.

18. The drug pricing system of claim 16 wherein the user interface further displays dosage information.

19. The drug pricing system of claim 14 further comprising not sending the electronic notification to the individual when past purchases indicate that the user prefers a higher insurance price over a lower second price from the PBM.

20. The drug pricing system of claim 14 further comprising automatically returning to the pharmacy system the lower of the insurance price and the second price from the PBM when past purchases indicate the user prefers a lowest drug price.

21. The drug pricing system claim 13 wherein the electronic notification is at least one of a text message, push notification, and an email, and the user device is a mobile device.
